(19) United States
(12) Patent Application Publication
(10) Pub. No.: US 2023/0045621 A1
(43) Pub. Date: Feb. 9, 2023

(54) METHODS AND SYSTEMS FOR DIAGNOSIS OF MYALGIC ENCEPHALOMYELITIS/CHRONIC FATIGUE SYNDROME (ME/CFS) FROM IMMUNE MARKERS

(71) Applicant: The Jackson Laboratory, Bar Harbor, ME (US)

(72) Inventor: Derya Unutmaz, Burlington, CT (US)

(73) Assignee: The Jackson Laboratory, Bar Harbor, ME (US)

(21) Appl. No.: 17/788,486

(22) PCT Filed: Dec. 18, 2020
(86) PCT No.: PCT/US2020/065985

§ 371 Date: Jun. 23, 2022

Related U.S. Application Data
(60) Provisional application No. 62/952,611, filed on Dec. 23, 2019.

(51) Int. Cl. G16H 50/20; G16B 40/20; G06N 20/20; G06N 7/00

(52) U.S. Cl. CPC G16H 50/20; G16B 40/20; G06N 20/20; G06N 7/005

(57) ABSTRACT

A method and system for developing a predictive model for diagnosis of myalgic encephalomyelitis/chronic fatigue syndrome (ME/CFS) in a human are disclosed. The method comprises receiving immune system data for each member of a population comprising healthy humans and humans with ME/CFS; extracting a set of features from the immune system data; and training a machine learning algorithm using the set of features to classify a human as healthy or having ME/CFS to obtain a predictive model. The system comprises a processor; and a memory storing computer executable instructions, which when executed by the processor cause the processor to perform operations of said method.

### CROSS-REFERENCE TO RELATED APPLICATIONS

This application claims priority to U.S. Provisional Application Ser. No. 62/952,611 filed Dec. 23, 2019, which is incorporated by reference herein in its entirety.

### STATEMENT OF GOVERNMENT SUPPORT

This invention was made with government support under RO1AI121920 and U54 NS1055 awarded by National Institutes of Health. The government has certain rights in the invention.

### BACKGROUND

This disclosure relates to immune biomarkers for myalgic encephalomyelitis/chronic fatigue syndrome (ME/CFS), methods and systems for developing predictive models for diagnosing ME/CFS by machine training a classifier algorithm using the immune biomarkers, and methods and systems for identifying ME/CFS patients using the predictive models.

Myalgic encephalomyelitis/chronic fatigue syndrome (ME/CFS) is a highly debilitating illness often characterized by symptoms such as post-exertional malaise or severe fatigue not alleviated by rest, muscle and joint pain, sleep problems, hypersensitivity to sensory stimuli, and gastrointestinal symptoms 1-3. ME/CFS is thought to afflict up to two million individuals in the US alone, with severe long-term disability and negative impacts on quality of life. The specific cause and biological basis of ME/CFS remain elusive. Lack of understanding of biological pathways leading to this syndrome is also a major impediment in developing specific therapies and reliable biomarker-based diagnostic tests.

While the causes of ME/CFS are likely to be multifactorial, many ME/CFS patients share a history of initial infection with agents, including viral (e.g. Epstein-Barr virus (EBV)) and bacterial (e.g. Lyme Disease) agents, which have been associated with triggering the disease. (Hickie, I. et al. BMJ 333, 575, doi:10.1136/bmj.38933.585764.AE (2006); Katz, B. Z., Shiraishi, Y., Mears, C. J., Binns, H. J. & Taylor, R. Pediatrics 124, 189-193, doi:10.1542/peds.2008-1879 (2009).) Mounting evidence in ME/CFS patients implicates a significant role for immunological abnormalities that are thought to contribute to disease progression and/or maintenance of the chronic symptomatic state.

The immune system appears to play an important role in the etiology or pathophysiology of ME/CFS. Studies of the immune system of ME/CFS subjects have revealed many abnormalities, including disruptions in the numbers and functions of T cell subsets, B cell and natural killer (NK) cells; changes in T-cell or innate cell cytokine secretion; changes in humoral immunity and inflammatory immune signaling; and higher frequencies of various autoantibodies. (Brenu, E. W. et al. Longitudinal investigation of natural killer cells and cytokines in chronic fatigue syndrome/myalgic encephalomyelitis. J Transl Med 10, 88, doi:10.1186/1479-5876-10-88 (2012); Curriu, M. et al. Screening NK-, B- and T-cell phenotype and function in patients suffering from Chronic Fatigue Syndrome. J Transl Med 11, 68, doi:10.1186/1479-5876-11-68 (2013); Brenu, E. W. et al. Role of adaptive and innate immune cells in chronic fatigue syndrome/myalgic encephalomyelitis. International immunology 26, 233-242, doi:10.1093/intimm/dxt068 (2014); Fletcher, M. A. et al. Biomarkers in chronic fatigue syndrome: evaluation of natural killer cell function and dipeptidyl peptidase IV/CD26. PLoS One 5, e10817, doi:10.1371/journal.pone.0010817 (2010); Tones-Harding, S., Sorenson, M., Jason, L. A., Maher, K. & Fletcher, M. A. Evidence for T-helper 2 shift and association with illness parameters in chronic fatigue syndrome (CFS). Bulletin of the IACFS/ME 16, 19-33 (2008); Broderick, G. et al. A formal analysis of cytokine networks in chronic fatigue syndrome. Brain Behav Immun 24, 1209-1217, doi:10.1016/j.bbi.2010.04.012 (2010); Bansal, A. S., Bradley, A. S., Bishop, K. N., Kiani-Alikhan, S. & Ford, B. Chronic fatigue syndrome, the immune system and viral infection. Brain Behav Immun 26, 24-31, doi:10.1016/j.bbi.2011.06.016 (2012); Prinsen, H. et al. Humoral and cellular immune responses after influenza vaccination in patients with chronic fatigue syndrome. BMC immunology 13, 71, doi:10.1186/1471-2172-13-71 (2012); Aspler, A. L., Bolshin, C., Vernon, S. D. & Broderick, G. Evidence of inflammatory immune signaling in chronic fatigue syndrome: A pilot study of gene expression in peripheral blood. Behavioral and brain functions: BBF 4, 44, doi:10.1186/1744-9081-4-44 (2008); Ortega-Hernandez, 0. D. & Shoenfeld, Y. Infection, vaccination, and autoantibodies in chronic fatigue syndrome, cause or coincidence? Annals of the New York Academy of Sciences 1173, 600-609, doi:10.1111/j.1749-6632.2009.04799.x (2009).)

In particular, T cells are responsible for orchestrating and modulating an optimal immune response, either through their effector or regulatory functions. Thus, perturbations in T cell subsets or in effector or regulatory functions during ME/CFS, can result in overall disruption or unwanted immune responses. (Lorusso, L. et al. Autoimmun Rev 8, 287-291, doi:10.1016/j.autrev.2008.08.003 (2009); Rivas, J. L., Palencia, T., Fernandez, G. & Garcia, M. Front Immunol 9, 1028, doi: 10.3389/fimmu.0.2018.01028 (2018).)

Currently, diagnosis of ME/CFS is based solely on clinical symptoms and runs a significant potential for diagnosis of false positives and false negatives. There is a need for improved diagnostic methods and biomarkers for diagnosis, particularly methods of diagnosis and biomarkers showing high sensitivity and specificity.

### BRIEF SUMMARY

A method and system for developing a predictive model for diagnosis of myalgic encephalomyelitis/chronic fatigue syndrome (ME/CFS) in a human are disclosed.

The method comprises receiving immune system data for each member of a population comprising healthy humans and humans with ME/CFS; extracting a set of features from the immune system data; and training a machine learning algorithm using the set of features to classify a human as healthy or having ME/CFS to obtain a predictive model.

The system comprises a processor; and a memory storing computer executable instructions, which when executed by the processor cause the processor to perform operations comprising: receiving immune system data for each member of a population comprising healthy humans and humans with ME/CFS; extracting a set of features from the immune system data; and training a machine learning algorithm using the set of features to classify a human as healthy or having ME/CFS to obtain a predictive model.

A method and system for diagnosing myalgic encephalomyelitis/chronic fatigue syndrome (ME/CFS) in a subject are also disclosed.

The method comprises: receiving immune system data of a subject; extracting a set of features from the immune system data; inputting the features to a machine-trained classifier, the machine trained classifier trained, at least in part, from training data comprising immune system data for a population comprising healthy humans and humans with ME/CFS; classifying, by application of the machine-trained classifier to the features, the subject as being healthy or having ME/CFS; and outputting the classification.

The system comprises a processor; and a memory storing computer executable instructions, which when executed by the processor cause the processor to perform operations comprising: comprises: receiving immune system data of a subject; extracting a set of features from the immune system data; inputting the features to a machine-trained classifier, the machine trained classifier trained, at least in part, from training data comprising immune system data for a population comprising healthy humans and humans with ME/CFS; classifying, by application of the machine-trained classifier to the features, the subject as being healthy or having ME/CFS; and outputting the classification.

The above described and other features are exemplified by the following figures and detailed description.

### BRIEF DESCRIPTION OF THE DRAWINGS

FIGS. 1a-g present frequency of main immune subsets in PBMC by flow cytometry was performed after staining and gating on (a) CD14+ (Monocytes) and CD19+ (B cells), and (b) CD3+ (T cells) and CD3-2B4+ (NK cells), and proportion of each subset frequency as a portion of PBMC are shown for each subject (right panels). (c) Frequencies of CD4+ and CD8+ T cells and their ratio were analyzed within CD3+ T cell gates. (d) Correlation of T cell subsets with age in ME/CFS patients and controls. (e) CD8+ T cell and CD4 to CD8 T cell ratio distribution in ages older and younger than 50 years in ME/CFS and controls, (f) Correlation of NK cells with age in ME/CFS patients and controls, (g) NK cell ratio distribution in groups based on ages older and younger than 50 years. Data from healthy controls (Healthy, n=90) and ME/CFS patients (ME/CFS, n=186) for a (left), from Healthy (n=91) and ME/CFS (n=190) for a (right), b (left), c, d, and e, from Healthy (n=91) and ME/CFS (n=189) for b (right), f, and g, and were compared by Mann-Whitney U test for non-parametric data, with exact p values, average (AVG) and median (MED) values are shown. Correlations of data were performed using nonparametric Spearman correlation, with exact rₛ and p value shown.

FIG. 2 presents graphs showing naïve and memory T cell subset frequencies in ME/CFS and healthy control PBMC. Naïve and memory T cell subsets were analyzed in PBMC by immune-staining and flow cytometry based on the expression of CD45RO and CCR7. (a) T cell subsets were analyzed in PBMC with CD45RO and CCR7 expression after gating on CD3+CD4+ (left) and CD3+CD8+ T cell subsets (right), which were then subdivided into CD45RO-CCR7+ or naïve (N), CD45RO+CCR7+ or central memory (CM), CD45RO+CCR7- or effector memory (EM), and CD45RO-CCR7- effector memory RA (EMRA) subsets as shown. (b) Proportions of naïve (N), central memory (CM), effector memory (EM) and effector memory RA (EMRA) T cell subsets were analyzed within CD3+CD4+ T cells and (c) CD3+CD8+ T cells in ME/CFS and healthy subjects. (d) The frequency of each subset was correlated to subject age for CD8+ T cells, by nonparametric Spearman correlation, with exact rₛ and p-value shown. (e) Analysis of CD8+ T cell subset frequencies in controls and ME/CFS patients that have been divided into two groups based on ages older and younger than 50 years. Data from healthy controls (Healthy, n=91) and ME/CFS patients (ME/CFS, n=190) for b-e, and groups were compared by Mann-Whitney test for non-parametric data, with exact p values shown, average (AVG) and median (MED) values are also shown. Correlations of data were performed using nonparametric Spearman correlation, with exact rₛ and p value shown.

FIG. 3 presents graphs showing the analysis and function of Th17 cell frequency and function in ME/CFS subjects. PBMC purified from patient or healthy control blood were stimulated as described in methods, surface stained, then fixed and permeabilized, and stained intracellularly for cytokine expression. (a) CD3+CD4+ cells were gated and the proportion of CD45RO+ and CCR6+ or CCR6− cells analyzed. (b) CD4+ memory (CD45RO+) T cells expressing IFNγ and/or IL-17 or after gating into CCR6+ and CCR6− T cells. (c) The frequency of IL-17 and/or IFNγ expression in CD4+CD45RO+ memory T cells in ME/CFS patient or control PBMC. (d) Same analysis was performed in PBMC after 6-day (d6) culture in IL-7. (e) Correlation of CD4+CD45RO+ memory T cells secreting IL-17 and/or IFNγ with subject age. Groups compared by nonparametric Spearman correlation, with exact rₛ and p-value shown. (f) Analysis of CD45RO+ memory IL-17 and/or IFNγ producing cells in control and ME/CFS patients divided into two groups based on ages older and younger than 50 years. Data from healthy controls (Healthy, n=80) and ME/CFS patients (ME/CFS, n=198) for c, from Healthy (n=90) and ME/CFS (n=195) for d, e, and f, and groups were compared by Mann-Whitney test for non-parametric data, with exact p-values, average (AVG) and median (MED) values shown. Correlations of data were performed using nonparametric Spearman correlation, with exact rₛ and p value shown.

FIG. 4 presents two graphs presenting the proportion of CCR6+ T cells in memory CD4+ cells after overnight culture (left) or 6 days in culture (right) (a) Proportion of CCR6+ T cells in memory CD4+ cells after day 1 (d1) or day 6 (d6) in culture in IL-7. (b) The frequency of CCR6+ T cells in memory CD4+ cells after day-1 culture correlated to subject age. Groups compared by nonparametric Spearman correlation, with exact rₛ and p-value shown. Analysis of CCR6+ T cells in memory CD4+ cells after day-1 culture in healthy control and ME/CFS patients divided into two age groups, (c) IL-17 and IFNγ expression in CD4+CCR6+CD45RO+ T cells in PBMC culture in IL-7 for 1 day or (d) for 6 days, post activation as described in methods. (e) Ratio of CD4+CCR6+ cells to IL-17+ or total IFNγ+ CD4+ memory cells calculated after cells after day-1 culture or (f) after 6 days in culture with IL-7. Data from healthy controls (Healthy, n=81) and ME/CFS patients (ME/CFS, n=198) for a (left), from Healthy (n=90) and ME/CFS (n=195) for a (right), from Healthy (n=80) and ME/CFS (n=197) for b, from Healthy (n=80) and ME/CFS (n=198) for c and e, from Healthy (n=90) and ME/CFS (n=196) for d, from Healthy (n=90) and ME/CFS (n=195) for f, and groups were compared by Mann-Whitney test for non-parametric data, with exact p values shown. Average (AVG) and median (MED) are also shown. Correlations of data were performed using nonparametric Spearman correlation, with exact rₛ and p value shown.

FIG. 5 presents graphs comparing Th17 cell frequency and function after culture in IL-7 for 6 days in ME/CFS and healthy subjects. PBMC from ME/CFS patients and healthy controls were cultured in IL-7 for 6 days and stimulated with PMA/ionomycin for 4 hours as described in methods. (a) Live CD4+ T cells were gated on different memory subsets based on CD161 expression (left) and the proportion of CD161+ cells within CD4+CD45RO+CCR6+ subset is shown for each subject (right). (b) Intracellular expression of IL-17 and IFNγ within CD4+CCR6+CD161+ and CD4+CCR6+CD161− T cell subsets. (c) The frequencies of IL-17 and IFNγ expressing cells within CD4+CD45RO+CCR6+CD161+ and (d) CD4+CD45RO+CCR6+CD161− cells were calculated and shown for individual study participants. (e) Analysis of IFNγ+IL4− (Th1 cells) and IFNγ−IL4+ (Th2 cells) in memory CD4+ cells after day 1 (d1) in culture in IL-7, and ratio of Th1 to Th2 cells. Data from healthy controls (n=90) and ME/CFS patients (n=196) for a (right), from Healthy (n=87) and ME/CFS (n=191) for c and d, from Healthy (n=90) and ME/CFS (n=198) for e, and groups were compared by Mann-Whitney test for non-parametric data, with exact p-values and average (AVG) and median (MED) values are shown. Correlations of data were performed using nonparametric Spearman correlation, with exact rₛ and p value shown.

FIG. 6 presents a graph of the in changes of Mucosal Associated Invariant T (MAIT) cell subset frequencies in ME/CFS PBMC compared to healthy controls. (a) MAIT cell subset frequencies were identified based on the co-expression of CD161 and Vα7.2, after gating within CD4+, CD8+ and CD4-CD8− (DN) T cells. (b) Analysis of the proportion of MAIT cells in each of these T cell subsets on day 0 (d0) and (c) 6 days (d6) in culture with IL-7. (d) Ratio of day 0 to day 6 MAIT cells was calculated for individual study participants. (e) The ratio of CD8+ MAIT to DN MAIT cells was calculated for day 0 (left) and day 6 (right). (f) Surface expression of CD27 was determined after gating for CD8+ MAIT and DN MAIT cell subsets as shown. (g) Analysis of the proportion of CD45RO+CD27− within CD8+ MAIT and DN MAIT cells in PBMC of ME/CFS and control subjects. Groups were compared by Mann-Whitney test for non-parametric data, with exact p values, average (AVG) and median (MED) values are shown. (h) The ratio of MAIT cell subset (CD8+ or DN separately) frequency at day 0 to day 6, was correlated with CD27− MAIT cell frequency (of total CD8+ MAIT cells). Data from healthy controls (Healthy, n=91) and ME/CFS patients (ME/CFS, n=190) for b, from Healthy (n=90) and ME/CFS (n=195) for c (left and middle), from Healthy (n=90) and ME/CFS (n=196) for c (right), from Healthy (n=90) and ME/CFS (n=186) ford (left), from Healthy (n=90) and ME/CFS (n=190) for d (middle), from Healthy (n=90) and ME/CFS (n=189) ford (right), from Healthy (n=91) and ME/CFS (n=190) for e (left), from Healthy (n=90) and ME/CFS (n=196) for e (right), from Healthy (n=91) and ME/CFS (n=190) for g, from Healthy (n=90) and ME/CFS (n=184) for h (left), from Healthy (n=60) and ME/CFS (n=108) for h (right), and groups were compared by Mann-Whitney test for non-parametric data, with exact p-values and average (AVG) and median (MED) values are shown. Correlations of data were performed using nonparametric Spearman correlation, with exact rₛ and p value shown.

FIG. 7 presents a graph of the in MAIT cell function after activation in ME/CFS PBMC compared to healthy controls. (a) PBMC were stimulated with combination of the cytokines IL-12+IL-15+IL-18 for 1 day as described in methods, and intracellularly stained for IFNγ and Granzyme A expression, which was analyzed after gating on MAIT (CD161+Vα7.2+) and non-MAIT (CD161−Vα7.2−) CD8+ T cells. (b) Proportion of IFNγ and (c) Granzyme A in CD8+ MAIT and non-MAIT cells from ME/CFS and control subjects. (d) The frequency of CD8+CD45RO+CD27− MAIT cells was correlated to CD8+ MAIT and non-MAIT IFNγ+ cells after stimulation with cytokine combination. Groups were compared by nonparametric Spearman correlation, with exact rₛ and p value shown in figures. (e) PBMC were cultured in IL-7 for 6 days (d6) then stimulated with PMA and lonomycin as described in methods. Frequency of IFNγ and IL-17A expression within MAIT and non-MAIT CD8+ T cells were compared between patient and control groups. (f) IFNγ and TNFα expression, after activation, in CD8+ MAIT, and CD8+CD45RO+ non-MAIT memory T cells. (g) Expression of IFNγ or IL-17+IFNγ cells within CD8+ MAIT cells in ME/CFS and control subjects. (h) Proportion of CD8+ or CD8+ memory (gated on CD45RO+) cells expressing IFNγ. Data from healthy controls (Healthy, n=91) and ME/CFS patients (ME/CFS, n=198) for b and c, from Healthy (n=91) and ME/CFS (n=185) ford (left), from Healthy (n=91) and ME/CFS (n=191) for d (right), from Healthy (n=91) and ME/CFS (n=188) for g (left), from Healthy (n=90) and ME/CFS (n=183) for g (right), from Healthy (n=90) and ME/CFS (n=196) for h, and groups were compared by Mann-Whitney test for non-parametric data, with exact p-values, average (AVG) and median (MED) values are shown. Correlations of data were performed using nonparametric Spearman correlation, with exact rₛ and p value shown.

FIG. 8 compares proportions of regulatory T cell (Treg) subsets in ME/CFS and healthy controls. (a) PBMC were stained with Foxp3 and Helios intracellularly and expression was analyzed after gating on CD4+ naïve (CD27+CD45RO−) and memory (CD45RO+) T cells as described in the methods. (b) Proportions of Tregs (Foxp3+Helios+) were calculated within CD4+ naïve and memory subsets in ME/CFS and healthy subjects. (c) CD4+ naïve and memory Tregs divided into two groups based on age younger and older than 50 years in all subjects. (d) Correlation of CD4+ naïve and memory Treg subset frequencies with subject age. (e) Correlation between Th17 cells and memory Tregs was performed by nonparametric Spearman correlation, with exact rₛ and p-value shown. The ratio of IL-17-expressing cells within Th17 subset (CCR6+) to memory Tregs were compared between ME/CFS subjects and controls. Data from healthy controls (Healthy, n=91) and ME/CFS patients (ME/CFS, n=197) for b, c, and d, from Healthy (n=80) and ME/CFS (n=197) for e, and groups compared by the Mann-Whitney test for non-parametric data, with exact p value, average (AVG) and median (MED) values are shown. Correlations of data were performed using nonparametric Spearman correlation, with exact rₛ and p value shown

FIG. 9 shows random forest machine learning algorithm results in identifying ME/CFS patients using set of the immune parameters analyzed. To generate a receiver operating characteristic (ROC) curve using random forest (RF) clustering algorithm, a training set with 231 samples (80% of total samples) was selected and the remaining data, corresponding to 58 samples (20% of total samples), was left as the test set. Missing values in the training and test sets were replaced by the corresponding median value in the training set. A K-fold cross-validation method was used (K=3) to tube the hyperparameters of the model and was trained using a distinct set of features as input; all 65 immune profile features, the 40 significantly different features, the top 10 significantly different features and the top 10 features that received the highest importance score are plotted.

### DETAILED DESCRIPTION

As further described herein, profound changes in CD8+ T cells, NK cells, Th17 and MAIT cell effector functions, and regulatory T (Treg) cell frequencies were identified in ME/CFS patients. In addition, use of a machine learning algorithm with the measured immune system markers resulted in the development of a predictive model to identify a subject as an ME/CFS patient with very high sensitivity and specificity.

Accordingly, a method and system for developing a predictive model for diagnosis of myalgic encephalomyelitis/chronic fatigue syndrome (ME/CFS) in a human are disclosed. The method comprises receiving immune system data for each member of a population comprising healthy humans and humans with myalgic encephalomyelitis/chronic fatigue syndrome (ME/CFS); extracting a set of features from the immune system data; and training a machine learning algorithm using the set of features to classify a human as healthy or as having ME/CFS to obtain a predictive model.

The system for developing a predictive model for diagnosis of ME/CFS in a human comprises a processor; and a memory storing computer executable instructions, which when executed by the processor cause the processor to perform operations comprising receiving immune system data for each member of a population comprising healthy humans and humans with myalgic encephalomyelitis/chronic fatigue syndrome (ME/CFS); extracting a set of features from the immune system data; and training a machine learning algorithm using the set of features to classify a human as healthy or as having ME/CFS to obtain a predictive model.

As used herein “machine learning” refers to using algorithms that give a computer system the ability to learn from data, identify patterns, and make predictions or decisions. The machine learning algorithm can be any suitable algorithm. For example, the machine learning algorithm can be a random forest classifier, a support vector machine, an artificial neural network, or a combination thereof.

The population of individuals comprises healthy humans and humans with myalgic encephalomyelitis/chronic fatigue syndrome (ME/CFS). A human with myalgic encephalomyelitis/chronic fatigue syndrome (ME/CFS) refers to an individual who has been diagnosed as having ME/CFS based on the criteria defined in Fukuda, K. et al. 1994 (“The chronic fatigue syndrome: a comprehensive approach to its definition and study. International Chronic Fatigue Syndrome Study Group,” Ann Intern Med 1994, 121, 953-959) or Carruthers B. M. et al. 2003 (Myalgic encephalomyelitis/chronic fatigue syndrome: clinical working case definition, diagnostic and treatment protocols. J Chronic Fatigue Syndr 2003; 11:7-115). The term “healthy human” refers to an individual with no known significant health problems.

The population can further comprise other groups of humans, for example humans with a medical condition or disease which may explain the presence of chronic fatigue. Examples of such conditions include untreated hypothyroidism, sleep apnea and narcolepsy, iatrogenic conditions such as side effects of medication, some types of malignancies, chronic cases of hepatitis B or C virus infection, a past or current diagnosis of a major depressive disorder with psychotic or melancholic features, alcohol or other substance abuse, severe obesity (body mass index≥45), and a combination thereof.

Immune system data received for each member of the population can be obtained by any suitable method. For example, the immune system data can be obtained from a database of such information, by measurement of the immune system biomarkers in blood samples from heathy subjects and known ME/CFS patients, or a combination thereof. The database of immune system data can be one maintained by a private source, such as a disease-specific research or advocacy organization, or by a public source, for example the National Institutes of Health. Measurement of the immune system biomarkers in blood samples from heathy subjects and known ME/CFS patients can be performed by any suitable method. Exemplary assays for measuring the immune system biomarkers by flow cytometry are described in the Examples.

The immune system data can include frequency of the main immune subsets in PBMCs, monocytes, B cells, T cells, and NK cells, and the proportion of each subset frequency as a portion of PBMC for each subject in the population. Such parameters can be determined by any suitable method, for example by flow cytometry performed after staining the cells for and gating on characteristic cell surface markers such as CD14+ (Monocytes), CD19+ (B cells), CD3+ (T cells), and CD3-2B4+ (NK cells). The immune system data can further include characterization of T cell subsets. For example, the immune system data can further include frequencies of CD4+ and CD8+ T cells, CD4− CD8− (double negative; “DN”) T cells, and/or the various possible ratios analyzed within CD3+ T cell gates.

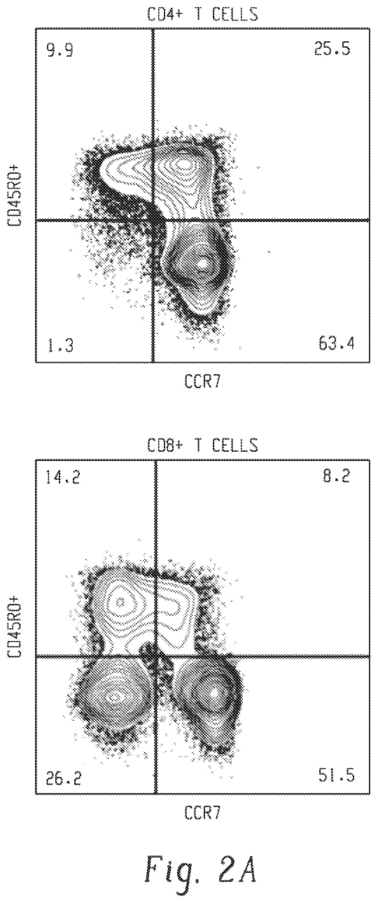

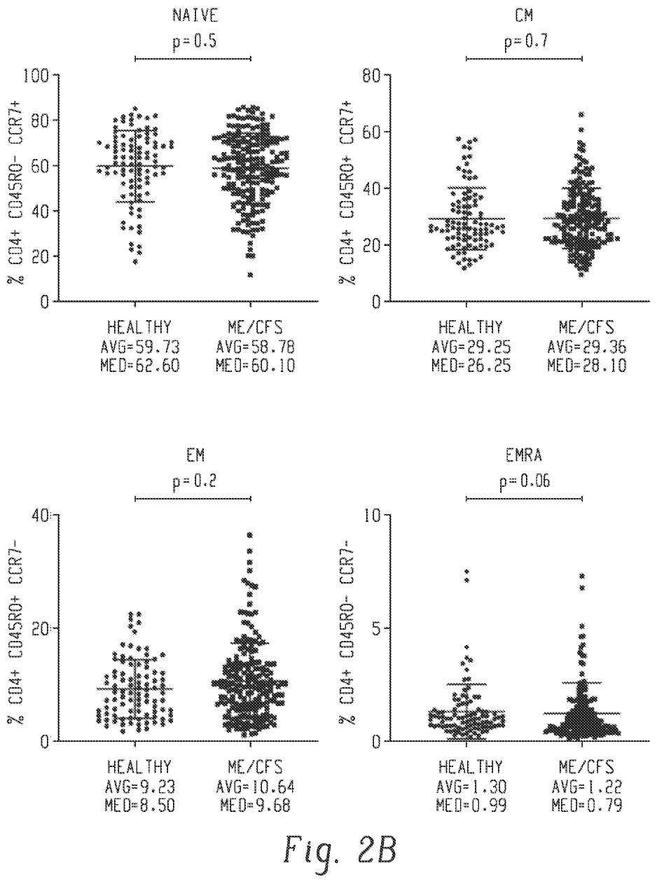

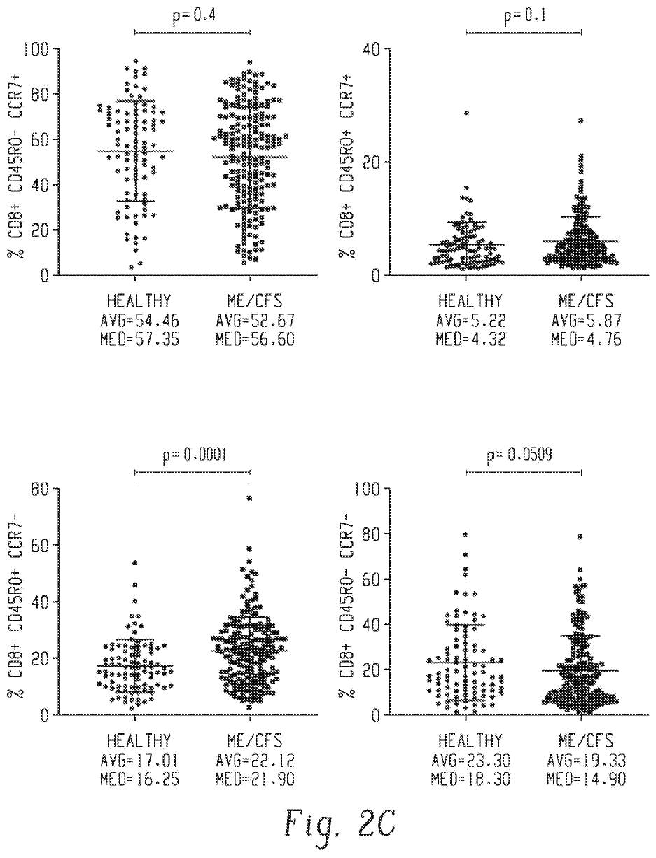

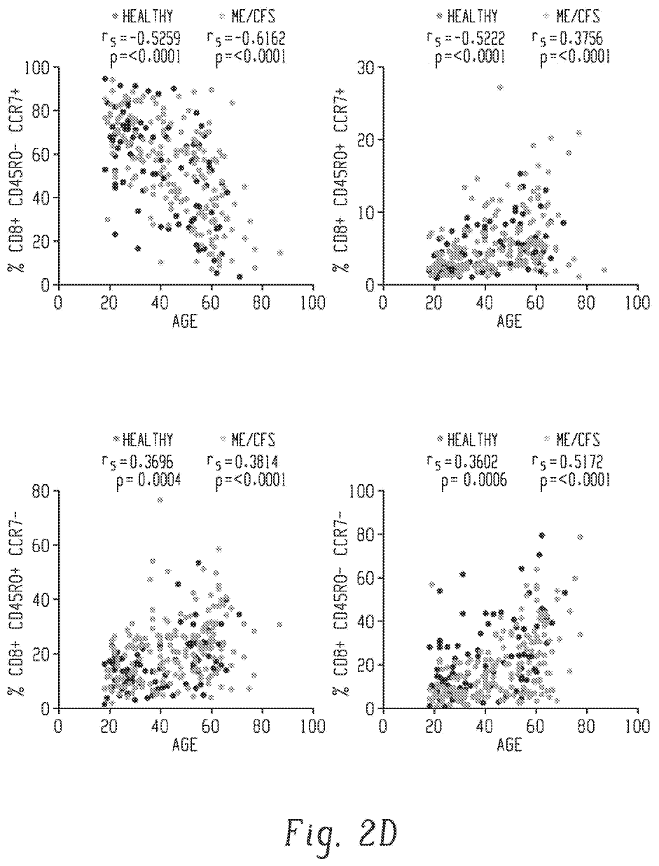

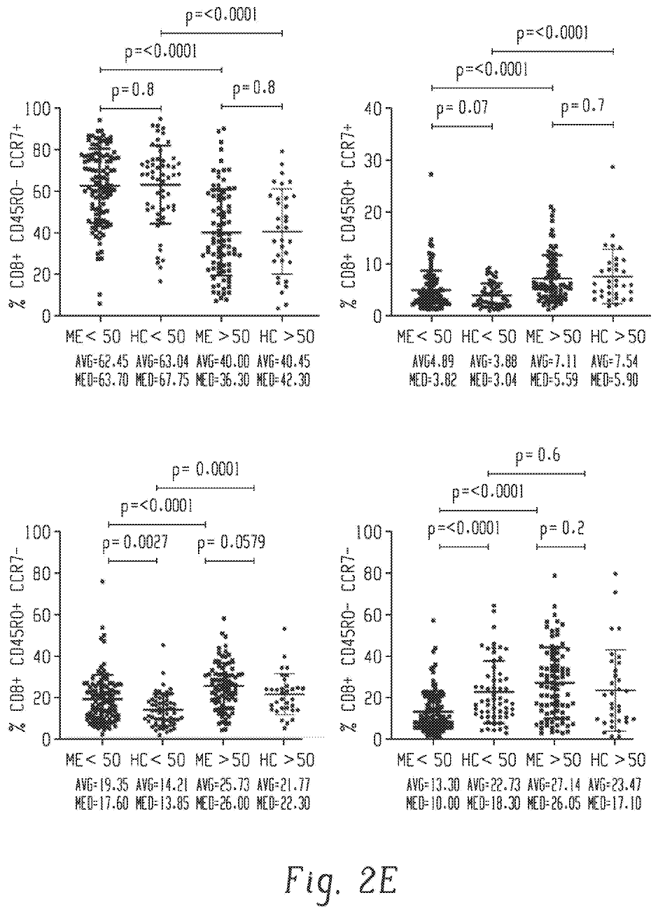

The immune system data can also further include characterization of the naïve and memory T cell subsets, which can be analyzed for example by flow cytometry after staining for CD45RO and CCR7 expression and gating on CD3+CD4+ or CD3+CD8+ T cell subsets, which can then be subdivided into CD45RO-CCR7+ or naïve (N), CD45RO+CCR7+ or central memory (CM), CD45RO+CCR7− or effector memory (EM), and CD45RO-CCR7-effector memory RA (EMRA) T cell subsets. Frequencies of each of these subsets, as well as proportion of each subset in the CD3+CD4+ or CD3+CD8+ T cell subset, respectively, can be determined as shown for example in FIG. 2.

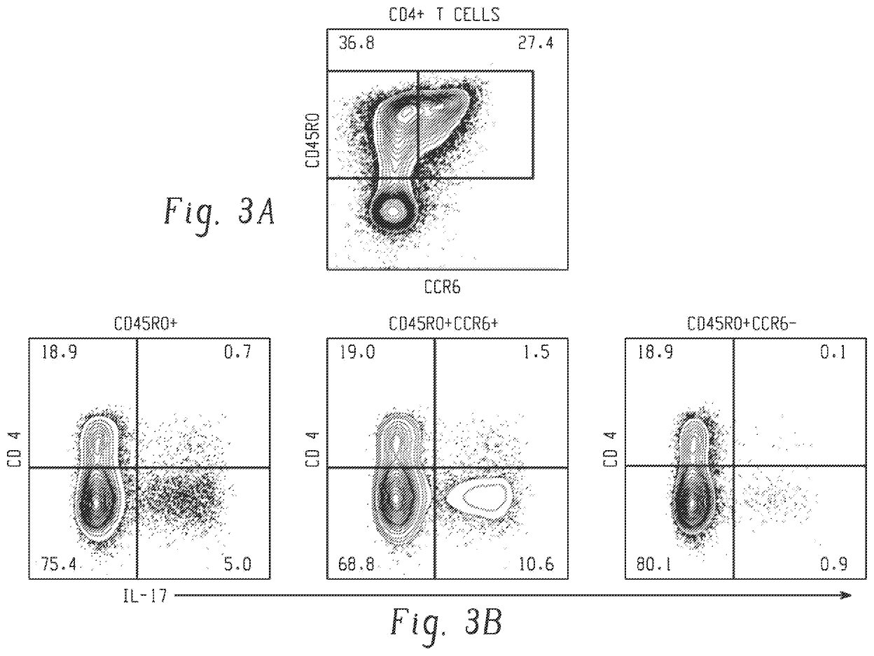

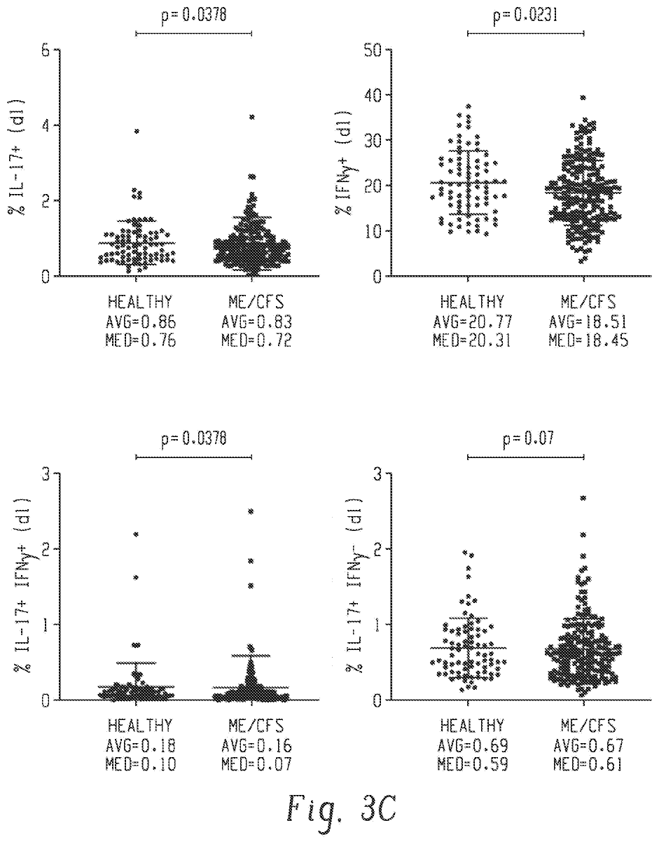

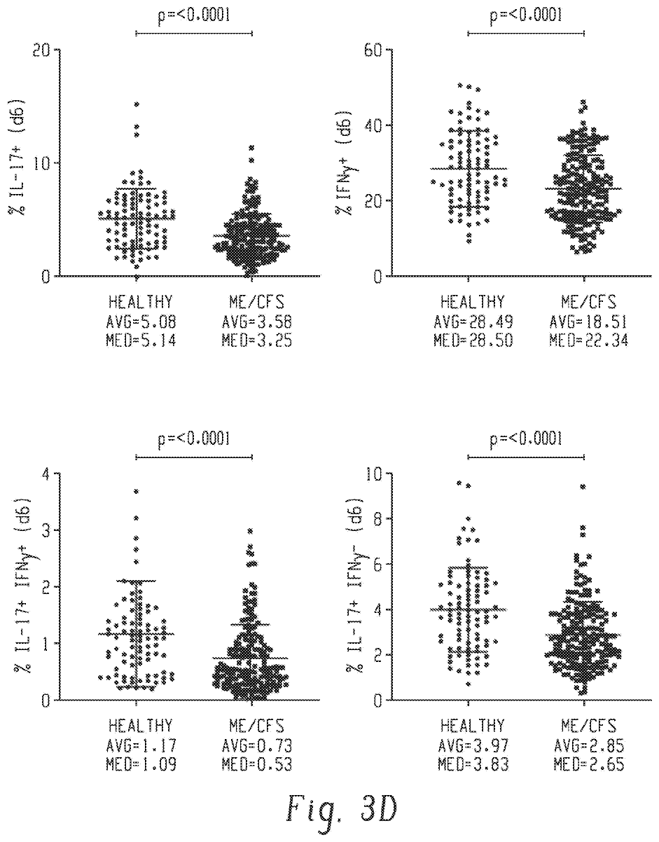

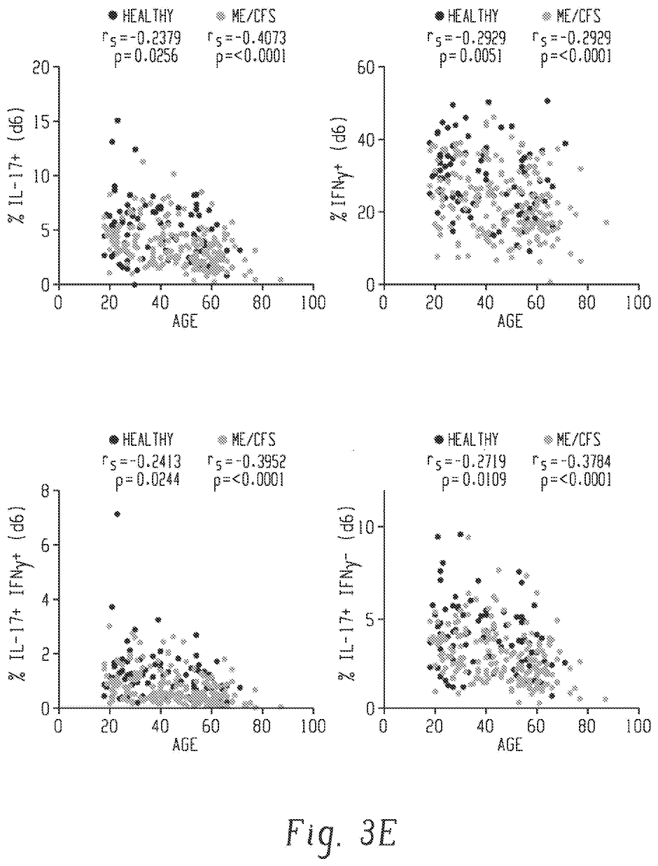

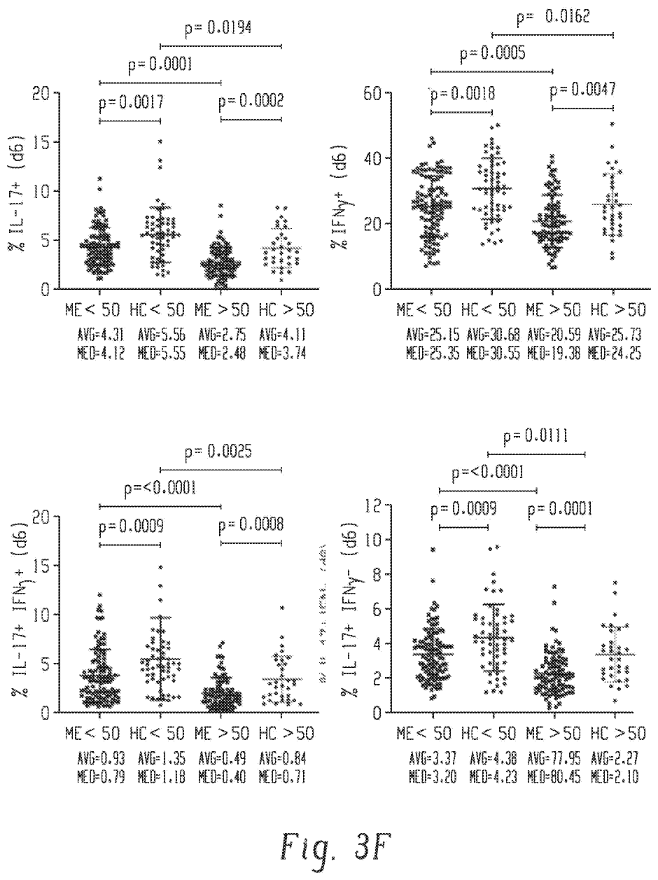

The immune system data can further comprise frequency and function of Th17 cells, which are an effector T cell subset that can produce IL-17 and play a role in response to bacterial infections or microbiota and are also linked to autoimmune diseases. Almost all of the subset of Th17 cells has a memory phenotype and also expresses the chemokine receptor CCR6, therefore Th17 cells can be detected by flow cytometry using CD3, CD4, CD45RO, and CCR6 expression (see for example, FIG. 3a). An exemplary method to analyze cytokine secretion from T cells comprises thawing frozen aliquots of PBMC and culturing one day (d1) in IL-7 to ensure cells have recovered from thawing and identify any dying cells; activating the cells with phorbol 12-myristate-13-acetate (PMA) and ionomycin as described in methods; staining the cells intracellularly for IL-17 and IFNγ expression; staining for expression of cell surface markers CD4, CD45RO, and CCR6; and gating on cell surface expression based on CD4+CD45RO+CCR6+ and CD4+CD45RO+CCR6− cells (see for example FIG. 3b), as previously described (Wan et al., 2011, Cytokine signals through PI-3 kinase pathway modulate Th17 cytokine production by CCR6+ human memory T cells. J Exp Med 208, 1875-1887). A portion of Th17 cells are poised to produce IL-17 or IL-22 only after priming with γc-cytokines (namely IL-2, IL-15 or IL-7) in culture, which reveal their full potential of their IL-17 secretion (Wan et al., 2011). Accordingly, PBMCs can be cultured in IL-7 to prime Th17 cells for IL-17 secretion, as previously described (Wan et al., 2011). Culturing PBMCs in 11-7 can be for 3 to 14 days, preferably 3 to 10 days, more preferably 5 to 7 days, yet more preferably 6 days (d6). PBMC are then stimulated using PMA and ionomycin as described elsewhere herein, followed by determination of expression of cytokines (for example, IL-17 or IFNγ) within the T cell subsets and also frequencies and/or proportions of the T cell subsets with the cytokine expression. Additionally, CD161 has been previously shown to divide CD4+CD45RO+CCR6+ T cells into subsets with differences in IL-17 and IFNγ secretion (Wan et al., 2011). Therefore CD161 expression can also be used to divide CCR6+ cells and determine IL-17 and IFNγ secretion.

In addition to determination of CD4+ memory T cells expressing IL-17, frequency of T cells expressing IFNγ (IFNγ+IL-4−) or IL-4 (IFNγ−IL-4+), defining Th1 and Th2 T cell subsets, respectively, can be determined in an analogous method.

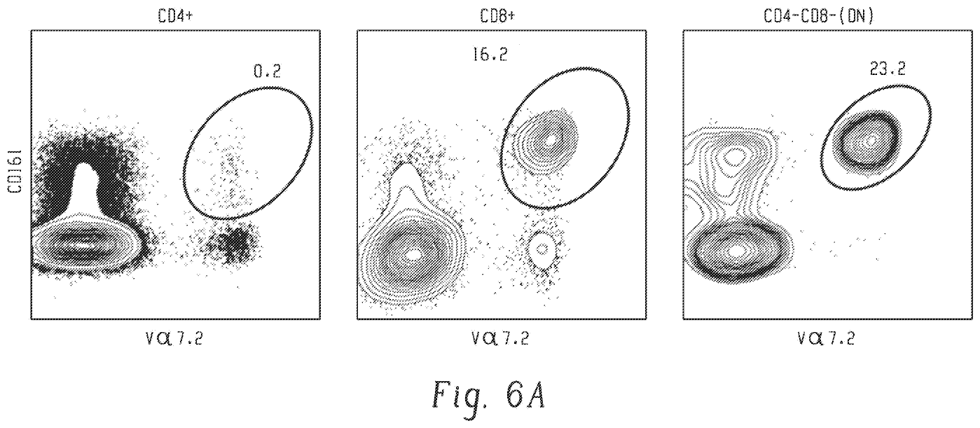

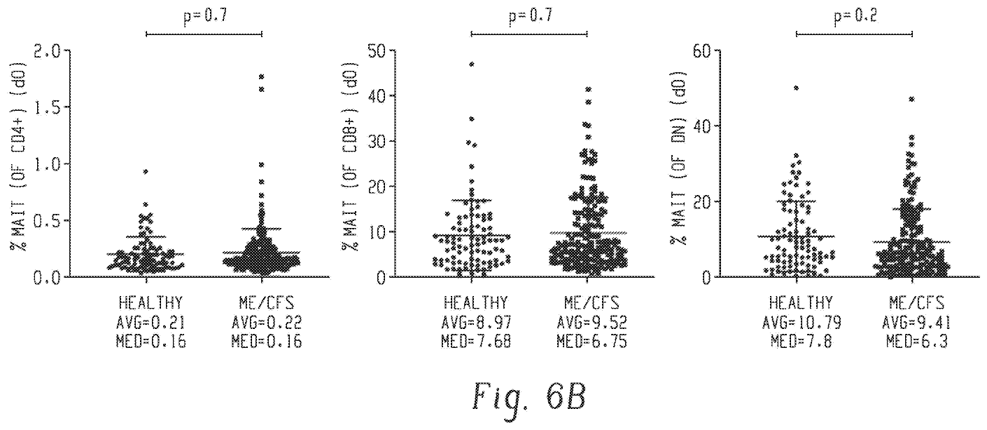

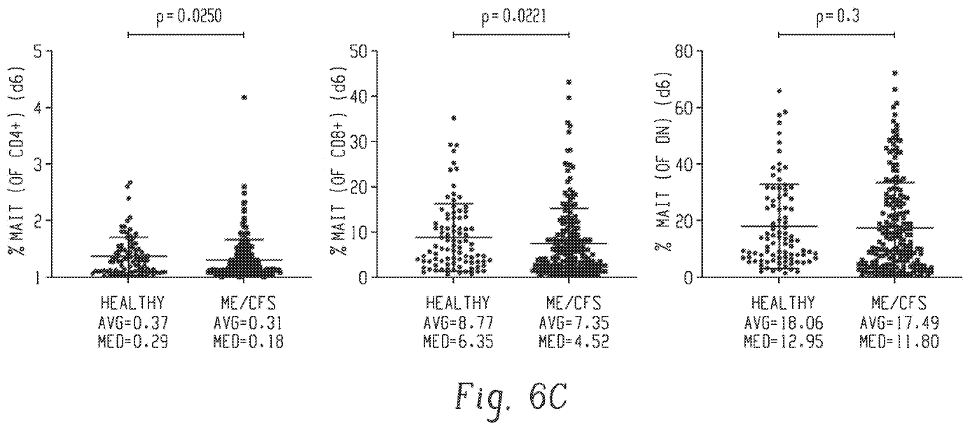

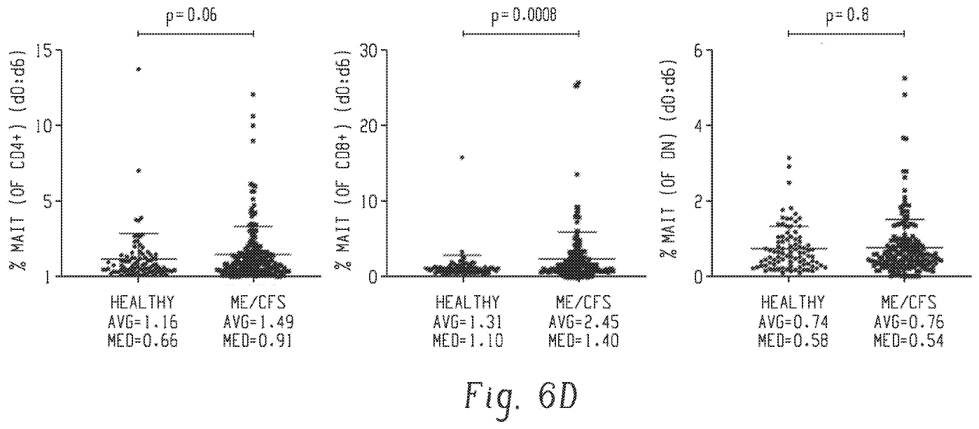

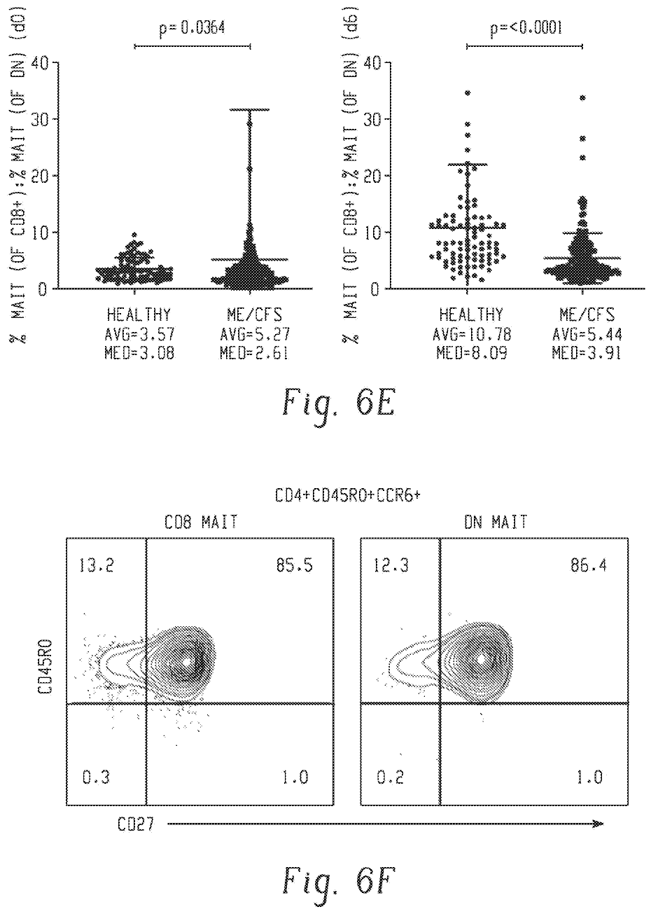

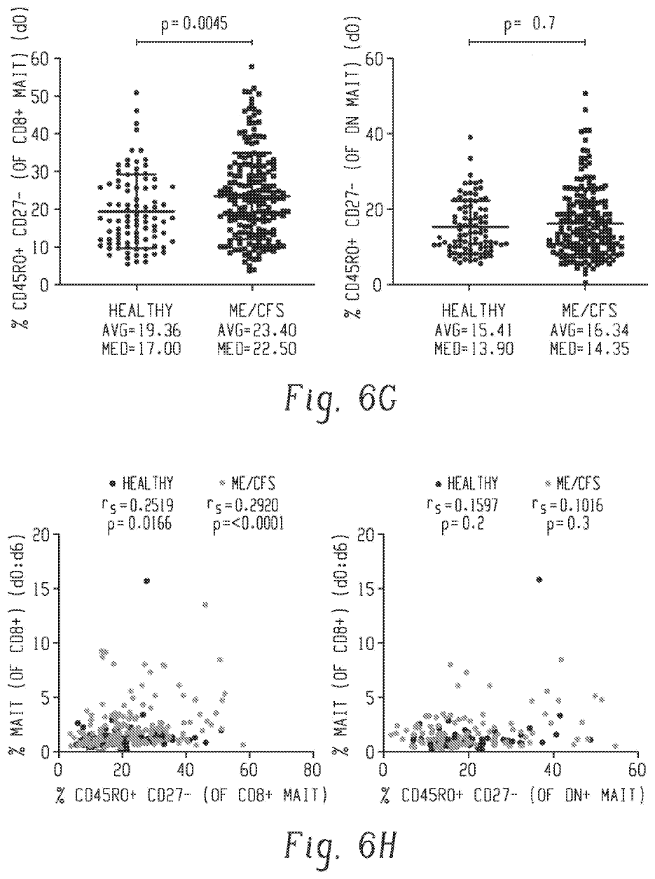

Mucosal-associated invariant T (MAIT) cells are a subset of the non-classical T cell population defined by an invariant T cell receptor that is triggered by riboflavin metabolites produced by bacteria, including commensal microbiota. To identify MAIT cells in PBMC, Vα7.2 and CD161 surface molecules can be used as previously described (Khaitan et al., 2016, HIV-Infected Children Have Lower Frequencies of CD8+ Mucosal-Associated Invariant T (MAIT) Cells that Correlate with Innate, Th17 and Th22 Cell Subsets. PLoS One 11, e0161786; Tastan et al., 2018, Tuning of human MAIT cell activation by commensal bacteria species and MR1-dependent T-cell presentation. Mucosal Immunol 11, 1591-1605). Frequency of MAIT cells within CD4+, CD8+ and CD4-CD8− (double negative or DN) T cell compartments can be determined (see for example FIG. 6a). Further, MAIT cell frequencies in PBMC after culture in the presence of IL-7, as described above, can be determined, as well as ratios of MAIT cell frequency at day 0 (d0) vs day y (“dy”, where y=3 to 14, preferably 3 to 10, more preferably 5 to 7, yet more preferably 6(“d6”)) after IL-7 culture. CD27 expression on MAIT cells is known to indicate a recently activated or differentiated subset, similar to other CD8 T cells (Dolfi and Katsikis, 2007, CD28 and CD27 costimulation of CD8+ T cells: a story of survival. Adv Exp Med Biol 590, 149-170; Grant et al., 2017, The role of CD27 in anti-viral T-cell immunity. Curr Opin Virol 22, 77-88). Therefore, the immune system data can also comprise frequency of CD27 expression in MAIT subsets.

The immune system data can also comprise parameters characterizing function of the MAIT cells. The PBMC can be stimulated with a cocktail of three cytokines, IL-12, IL-15, and IL18, since this combination has been uniquely shown to induce expression of IFNγ from MAIT cells (Ussher et al., 2014, CD161++CD8+ T cells, including the MAIT cell subset, are specifically activated by IL-12+IL-18 in a TCR-independent manner. Eur J Immunol 44, 195-203; Salou et al., 2017, MAIT cells in infectious diseases. Curr Opin Immunol 48, 7-14). Expression of IFNγ and/or Granzyme A expression can be used to evaluate response of CD8+ MAIT and CD8+ non-MAIT cells in PBMC to stimulation with a IL-12+IL-15+IL18 cocktail. MAIT cells have also been shown to express IL-17, similar to Th17 cells (Salou, M., Franciszkiewicz, K., and Lantz, O. (2017). MAIT cells in infectious diseases. Curr Opin Immunol 48, 7-14). Therefore the immune system data can also comprise frequency of production of IL-17 and IFNγ from MAIT cells in response to PMA and ionomycin stimulation in cultured PBMCs. The immune system data can also comprise frequency of IFNγ and TNFα secretion from CD8+ MAIT and CD8+ non-MAIT CD45RO+ (memory) T cells after PBMC culture with IL-7, as described elsewhere herein.

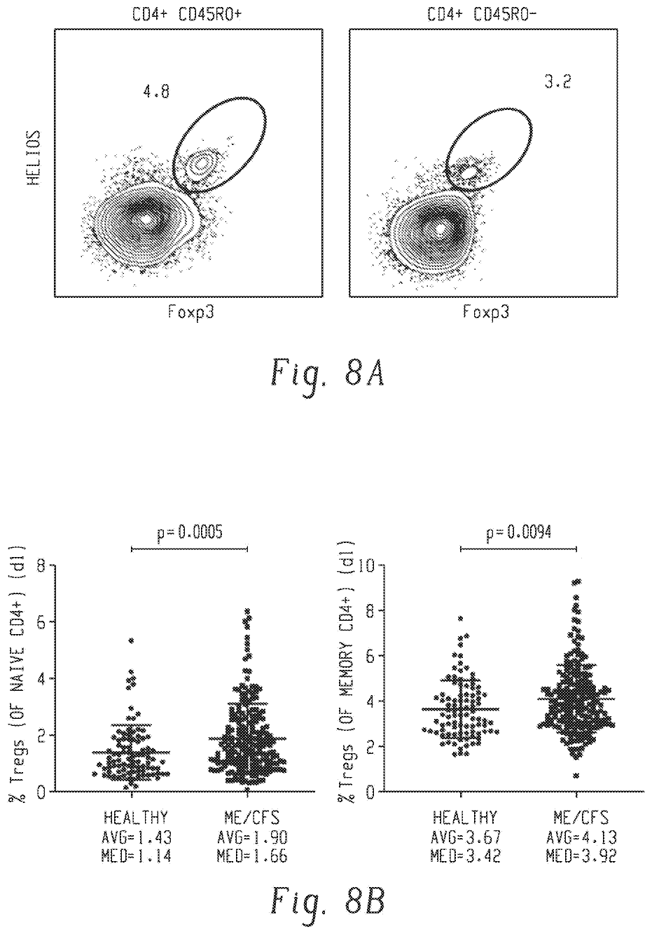

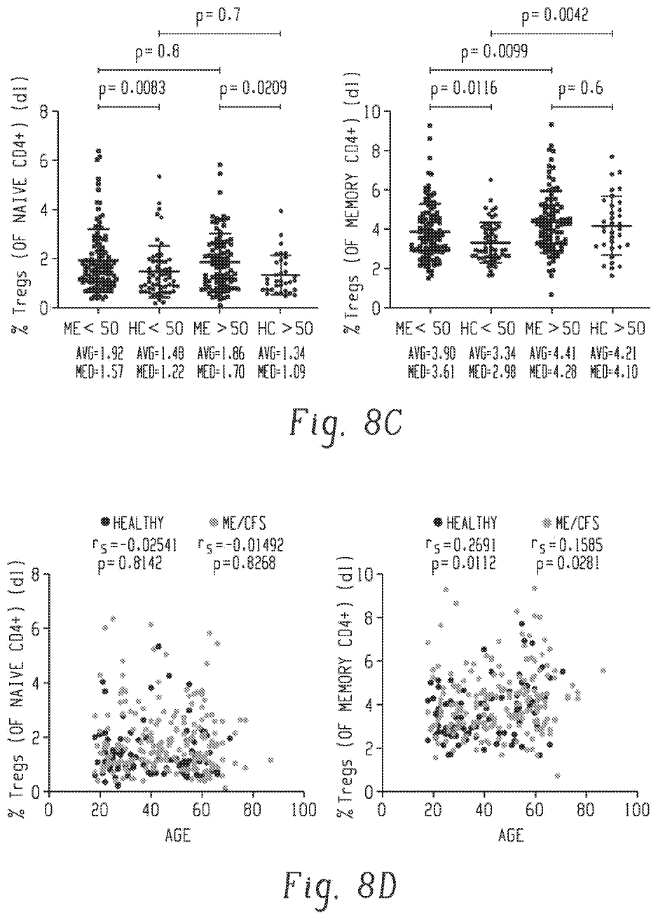

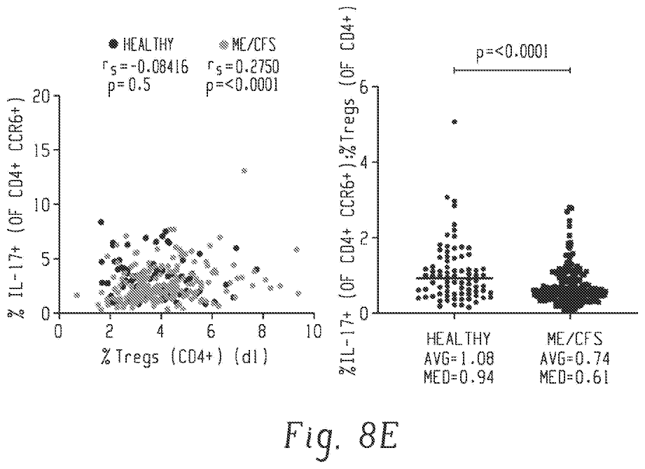

Regulatory T (Tregs) cells are critical in controlling autoreactive or excessive immune responses. Further, the ratio of Th17 cells to Tregs is an important feature that is perturbed during chronic inflammatory conditions or autoimmune diseases. Thus the immune profile data can further comprise frequency of Tregs and the ratio of Th17 (CCR6+IL-17-secreting cells) to Tregs. Foxp3 and Helios can be used as markers to assess Treg cell frequencies within both naïve and memory CD4+ T cells, as previously described (Mercer et al., 2014, Differentiation of IL-17-producing effector and regulatory human T cells from lineage-committed naive precursors. J Immunol 193, 1047-1054) (see for example FIG. 8a).

Extracting a set of features from the immune system data can be performed by any suitable method. The features extracted from the immune system data can comprise at least one of the features listed in Table 2 below. The features extracted from the immune system data can comprise all of the features listed in Table 2. The number of features, and which features, in Table 2 are extracted from the immune system data can be selected to optimize performance of the predictive model.

TABLE 2
Immune profile features determined for the ME/CFS patients and healthy controls

| No. | Feature |
|---|---|
| 1 | % CD3+ |
| 2 | % CD8+ |
| 3 | % CD4+ |
| 4 | CD4:CD8 |
| 5 | % CD4− CD8− |
| 6 | % CD4+ CD45RO+ CCR7+ |
| 7 | % CD4+ CD45RO− CCR7+ |
| 8 | % CD4+ CD45RO+ CCR7− |
| 9 | % CD4+ CD45RO− CCR7− |
| 10 | % CD8+ CD45RO+ CCR7+ |
| 11 | % CD8+ CD45RO− CCR7+ |
| 12 | % CD8+ CD45RO+ CCR7− |
| 13 | % CD8+ CD45RO− CCR7− |
| 14 | % CD45RO+ CD27+ (of DN) (d 0) |
| 15 | % CD45RO− CD27− (of DN) (d 0) |
| 16 | % CD45RO+ CD27− (of DN) (d 0) |
| 17 | % CD45RO+ CD27− (of CD8+ MAIT) (d 0) |
| 18 | % MAIT (of CD4+) (d 0) |
| 19 | % MAIT (of CD8+) (d 0) |
| 20 | % MAIT (of DN) (d 0) |
| 21 | % MAIT (of CD8+):% MAIT (of DN) (d 0) |
| 22 | CD4+ total memory % IL−17+ IFNγ+ (dy, where y = 3 to 14, preferably 3 to 10, more |
| | preferably 5-7, yet more preferably y = 6) |
| 23 | CD4+ total memory % IL-17+ IFNγ− (dy, where y = 3 to 14, preferably 3 to 10, more |
| | preferably 5-7, yet more preferably y = 6) |
| 24 | CD4+ total memory % IL-17+ (dy, where y = 3 to 14, preferably 3 to 10, more preferably |
| | 5-7, yet more preferably y = 6) |
| 25 | CD4+ total memory % IFNγ+ (dy, where y = 3 to 14, preferably 3 to 10, more preferably |
| | 5-7, yet more preferably y = 6) |
| 26 | CD4+ RO+ % IL-17+ IFNγ+ (of CCR6+) (dy, where y = 3 to 14, preferably 3 to 10, more |
| | preferably 5-7, yet more preferably y = 6) |
| 27 | CD4+ RO+ % IL-17+ IFNγ− (of CCR6+) (dy, where y = 3 to 14, preferably 3 to 10, more |
| | preferably 5-7, yet more preferably y = 6) |
| 28 | CD4+ RO+ % IL-17− IFNγ+ (of CCR6+) (dy, where y = 3 to 14, preferably 3 to 10, more |
| | preferably 5-7, yet more preferably y = 6) |
| 29 | CD4+ RO+ % IL-17+ (of CCR6+) (dy, where y = 3 to 14, preferably 3 to 10, more |
| | preferably 5-7, yet more preferably y = 6) |
| 30 | CD4+ RO+ % IFNγ+ (of CCR6+) (dy, where y = 3 to 14, preferably 3 to 10, more |
| | preferably 5-7, yet more preferably y = 6) |
| 31 | % IFNγ+ (of memory CD4+) (dy, where y = 3 to 14, preferably 3 to 10, more preferably |
| | 5-7, yet more preferably y = 6) |
| 32 | CD4+ CD45RO+ CCR6+ CD161+ % IL-17+ IFNγ+ (dy, where y = 3 to 14, preferably 3 |
| | to 10, more preferably 5-7, yet more preferably y = 6) |
| 33 | CD4+ CD45RO+ CCR6+ CD161+ % IL-17+ IFNγ− (dy, where y = 3 to 14, preferably 3 |
| | to 10, more preferably 5-7, yet more preferably y = 6) |
| 34 | CD4+ CD45RO+ CCR6+ CD 161+ % IL-17− IFNγ+ (dy, where y = 3 to 14, preferably 3 |
| | to 10, more preferably 5-7, yet more preferably y = 6) |
| 35 | CD4+ CD45RO+ CCR6+ CD161− % IL-17+ IFNγ+ (dy, where y = 3 to 14, preferably 3 |
| | to 10, more preferably 5-7, yet more preferably y = 6) |
| 36 | CD4+ CD45RO+ CCR6+ CD161− % IL-17+ IFNγ− (dy, where y = 3 to 14, preferably 3 |
| | to 10, more preferably 5-7, yet more preferably y = 6) |
| 37 | CD4+ CD45RO+ CCR6+ CD161− % IL-17− IFNγ+ (dy, where y = 3 to 14, preferably 3 |
| | to 10, more preferably 5-7, yet more preferably y = 6) |
| 38 | % MAIT (of CD4+) (dy, where y = 3 to 14, preferably 3 to 10, more preferably 5-7, yet |
| | more preferably y = 6) |
| 39 | % MAIT (of CD8+) (dy, where y = 3 to 14, preferably 3 to 10, more preferably 5-7, yet |
| | more preferably y = 6) |
| 40 | % MAIT (of DN) (dy, where y = 3 to 14, preferably 3 to 10, more preferably 5-7, yet more |
| | preferably y = 6) |
| 41 | % MAIT (of CD8+): % MAIT (of DN) (dy, where y = 3 to 14, preferably 3 to 10, more |
| | preferably 5-7, yet more preferably y = 6) |
| 42 | % IL-17+ IFNγ+ (of CD8+ MAIT) (dy, where y = 3 to 14, preferably 3 to 10, more |
| | preferably 5-7, yet more preferably y = 6) |
| 43 | % IFNγ+ (of CD8+ MAIT) (dy, where y = 3 to 14, preferably 3 to 10, more preferably 5- |
| | 7, yet more preferably y = 6) |
| 44 | % IL-17+ (of CD8+ MAIT) (dy, where y = 3 to 14, preferably 3 to 10, more preferably 5- |
| | 7, yet more preferably y = 6) |
| 45 | % TNFa (of CD8+ MAIT) (dy, where y = 3 to 14, preferably 3 to 10, more preferably 5- |
| | 7, yet more preferably y = 6) |
| 46 | % MAIT (of CD4+) (d 0:dy, where y = 3 to 14, preferably 3 to 10, more preferably 5-7, |
| | yet more preferably y = 6) |
| 47 | % MAIT (of CD8+) (d 0:dy, where y = 3 to 14, preferably 3 to 10, more preferably 5-7, |
| | yet more preferably y = 6) |
| 48 | % MAIT (of DN) (d 0:dy, where y = 3 to 14, preferably 3 to 10, more preferably 5-7, yet |
| | more preferably y = 6) |
| 49 | % CCR6+ (of memory CD4+) (d 1) |
| 50 | CD4+ total memory % IL-17+ (d 1) |
| 51 | CD4+ RO+ % IL-17+ IFNγ+ (d 1) |
| 52 | CD4+ RO+ % IL-17+ IFNγ− (d 1) |
| 53 | CD4+ RO+ % IL-17+ (d 1) |
| 54 | CD4+ RO+ % IFNγ+ (d 1) |
| 55 | CD4+ RO+ % IL-17+ IFNγ+ (of CCR6+) (d 1) |
| 56 | CD4+ RO+ % IL-17+ IFNγ− (of CCR6+) (d 1) |
| 57 | CD4+ RO+ % IL-17+ (of CCR6+) (d 1) |
| 58 | CD4+ RO+ % IFNγ+ (of CCR6+) (d 1) |
| 59 | % IFNγ+ (of memory CD4+) (d 1) |
| 60 | % IFNγ+ (of CD8+ MAIT) (d 1) |
| 61 | % GranzymeA+ (of CD8+ MAIT) (d 1) |
| 62 | % Tregs (of naïve CD4+) (d 1) |
| 63 | % FOXP3+ (of naïve CD4+) (d 1) |
| 64 | % Tregs (of memory CD4+) (d 1) |
| 65 | % FOXP3+ (of memory CD4+) (d 1) |

In Table 2, “dx”, where x is a number from 0 to 14, indicates the immune system parameter is determined in a subject's isolated peripheral blood mononuclear cells (PBMCs) x days after culturing in a suitable medium. For example “d0” indicates the immune system parameter was determined in PBMCs prior to culturing, “d1” indicates the immune system parameter was determined in PBMCs after culturing for one day, and “d6” indicates the immune system parameter was determined in PBMCs after culturing for six days.

The PBMCs used in the measurement of the immune system properties can be freshly isolated or thawed after cryopreservation of the isolated PBMCs at liquid nitrogen temperatures. Isolation of PBMCs from a subject's blood sample can be performed by any suitable method. One exemplary method is to isolate the PBMCs from a blood sample, such as a heparinized blood sample, by density gradient centrifugation. Suitable density gradient media are sold commercially, such as FICOLL-PAQUE PLUS (GE Helathcare).

Suitable media for culturing PBMCs are known. An exemplary medium is RPMI 1640 medium (RPMI) plus 10% Fetal Bovine Serum (FBS) and 1% penicillin/streptomycin. As is known in the art, the culture medium can be supplemented with various cytokines, such as IL-2, IL-15, IL-12, IL-18, IL-7, at a suitable concentration to permit measurement of particular subsets of regulatory T cells (Tregs) and/or to permit measurement of particular surface or intracellular cytokines on immune cells at selected time points during culture of the PBMCs.

The features extracted from the immune system data can comprise at least one of the features listed in Table 3 below. Table 3 is a subset of the Table 2 features showing statistically significant difference between healthy and ME/CFS patients in an exemplary population of 231 humans (73 healthy; 158 ME/CFS) after adjustment for a false discovery rate. The features extracted from the immune system data can comprise at least the first ten features listed in Table 3. The features extracted from the immune system data can comprise all of the features listed in Table 3. The number of features, and which features, in Table 3 are extracted from the immune system data can be selected to optimize performance of the predictive model.

TABLE 3
Immune profile features significantly different between healthy controls and ME/CFS patients.

| No. | Feature | Raw p* | Adjusted p* |
|---|---|---|---|
| 1 | MAIT cells % of CD8+ to MAIT % of DN cells (dy, where y = 3 to | 1.48e−12 | 9.62e−11 |
| | 14, preferably 3 to 10, more preferably 5-7, yet more preferably y = | | |
| | 6) | | |
| 2 | Granzyme A+ % of CD8+ MAIT cells (d 1) | 2.16e−09 | 7.04e−08 |
| 3 | IL-17+ % of CD4+CD45O+ memory (dy, where y = 3 to 14, | 1.11e−07 | 2.40e−06 |
| | preferably 3 to 10, more preferably 5-7, yet more preferably y = 6) | | |
| 4 | IL-17+ IFNγ− of CD4+CD45RO+ memory (dy, where y = 3 to 14, | 4.35e−07 | 7.07e−06 |
| | preferably 3 to 10, more preferably 5-7, yet more preferably y = 6) | | |
| 5 | IL-17+IFNγ+ % of CD4+CD45RO+ memory (dy, where y = 3 to | 8.70e−07 | 1.13e−05 |
| | 14, preferably 3 to 10, more preferably 5-7, yet more preferably y = | | |
| | 6) | | |
| 6 | IL-17+ % of CD4+CD45RO+CCR6+ (dy, where y = 3 to 14, | 1.31e−06 | 1.42e−05 |
| | preferably 3 to 10, more preferably 5-7, yet more preferably y = 6) | | |
| 7 | IL-17+IFNγ+ % of CD4+CD45RO+CCR6+ (dy, where y = 3 to 14, | 1.70e−06 | 1.58e−05 |
| | preferably 3 to 10, more preferably 5-7, yet more preferably y = 6) | | |
| 8 | IFNγ+ % of CD4+CD45RO+ memory (dy, where y = 3 to 14, | 7.53e−06 | 6.12e−05 |
| | preferably 3 to 10, more preferably 5-7, yet more preferably y = 6) | | |
| 9 | IFNγ+ % of CD8+ MAIT cells (d 1) | 1.01e−05 | 7.33e−05 |
| 10 | IL-17+IPNγ− % of CD4+CD45RO+CCR6+ (dy, where y = 3 to 14, | 1.32e−05 | 7.36e−05 |
| | preferably 3 to 10, more preferably 5-7, yet more preferably y = 6) | | |
| 11 | MAIT cell ratio (d 0:dy, where y = 3 to 14, preferably 3 to 10, more | 3.19e−05 | 0.00018 |
| | preferably 5-7, yet more preferably y = 6) % of CD8+ | | |
| 12 | IFNγ+ % of CD4+CD45RO+ memory (dy, where y = 3 to 14, | 5.21e−05 | 0.00028 |
| | preferably 3 to 10, more preferably 5-7, yet more preferably y = 6) | | |
| 13 | IL-17+IFNγ+ % of CD4+CD45RO+CCR6+CD161− (dy, where y = | 9.69e−05 | 0.00048 |
| | 3 to 14, preferably 3 to 10, more preferably 5-7, yet more preferably | | |
| | y = 6) | | |
| 14 | CD8+CD45RO+CCR7− % of CD8+ | 0.00011 | 0.00053 |
| 15 | Tregs % of naïve CD4+ (d 1) | 0.00019 | 0.00086 |
| 16 | CCR6+ % of memory CD4+ (d 1) | 0.00048 | 0.0019 |
| 17 | IFNγ+ % of CD4+CD45RO+CCR6+ (dy, where y = 3 to 14, | 0.00078 | 0.0030 |
| | preferably 3 to 10, more preferably 5-7, yet more preferably y = 6) | | |
| 18 | IL-17+ % of CD4+CD45RO+CCR6+ (d 1) | 0.0019 | 0.0064 |
| 19 | IL-17+ % of CD4+CD45RO+ (d 1) | 0.0019 | 0.0064 |
| 20 | IFNγ+ % of memory CD4+ (d 1) | 0.0019 | 0.0064 |
| 21 | FOXP3+ % of memory CD4+ (d 1) | 0.0021 | 0.0066 |
| 22 | IL-17+IFNγ+ % of CD4+CD45RO+CCR6+ (d 1) | 0.0022 | 0.0067 |
| 23 | CD4+CD45RO+CCR6+CD161− % IL-17+IPNγ− (dy, where y = 3 | 0.0027 | 0.0075 |
| | to 14, preferably 3 to 10, more preferably 5-7, yet more preferably | | |
| | y = 6) | | |
| 24 | CD45RO+CD27− % of CD8+ MAIT | 0.0028 | 0.0075 |
| 25 | CD8+ % of CD3+ | 0.0033 | 0.0086 |
| 26 | Tregs % of CD4+ memory (d 1) | 0.0043 | 0.011 |
| 27 | CD4+ to CD8+ T cell ratio | 0.0048 | 0.011 |
| 28 | IL-17+IPNγ− % of CD4+CD45RO+CCR6+ (d 1) | 0.005 | 0.011 |
| 29 | IL-17+IFNγ+ % of CD4+CD45RO+CCR6+CD161+ (dy, where | 0.0051 | 0.011 |
| | y = 3 to 14, preferably 3 to 10, more preferably 5-7, yet more | | |
| | preferably y = 6) | | |
| 30 | CD4+ RO+ % IL-17− IFNγ+ (of CCR6+) (dy, where y = 3 to 14, | 0.006 | 0.013 |
| | preferably 3 to 10, more preferably 5-7, yet more preferably y = 6) | | |
| 31 | MAIT ratio (d 0:dy, where y = 3 to 14, preferably 3 to 10, more | 0.0092 | 0.019 |
| | preferably 5-7, yet more preferably y = 6) % of CD4+ | | |
| 32 | CD4+ % of CD3+ | 0.011 | 0.023 |
| 33 | IL-17+IFNγ+ % of CD8+ MAIT cells (dy, where y = 3 to 14, | 0.012 | 0.023 |
| | preferably 3 to 10, more preferably 5-7, yet more preferably y = 6) | | |
| 34 | MAIT % of CD4+ (dy, where y = 3 to 14, preferably 3 to 10, more | 0.013 | 0.024 |
| | preferably 5-7, yet more preferably y = 6) | | |
| 35 | MAIT % of CD8+ (dy, where y = 3 to 14, preferably 3 to 10, more | 0.013 | 0.024 |
| | preferably 5-7, yet more preferably y = 6) | | |
| 36 | CD8+ MAIT ratio to DN MAIT cells (d 0) | 0.015 | 0.027 |
| 37 | IL-17IFNγ+ % of CD4+CD45RO+ (d 1) | 0.016 | 0.028 |
| 38 | IFNγ+ % of CD4+CD45RO+ (d 1) | 0.017 | 0.029 |
| 39 | CD45RO+CD27− % of DN T cells (d 0) | 0.018 | 0.030 |
| 40 | CD8+CD45RO−CCR7− % of CD8+ | 0.021 | 0.035 |

*Raw p-value: Student's t-test or Mann-Whitney U test. Adj. p-value: adjusted p-values after 5% false discovery rate correction.

In certain embodiments, the features extracted from the immune system data can comprise at least one of the features listed in Table 4 below. Table 4 is a subset of the Table 3 features that received the highest importance score in a RF classifier model trained using all of the Table 4 features for an exemplary population of 231 humans (73 healthy; 158 ME/CFS). The features extracted from the immune system data can comprise all of the features listed in Table 4. The number of features, and which features, in Table 4 are extracted from the immune system data can be selected to optimize performance of the predictive model.

TABLE 4
The 10 features with the highest importance score in an exemplary predictive model

| Immune Features | Importance score* | Adj. p value |
|---|---|---|
| MAIT % of CD8+ to MAIT % of DN ratio(dy, where y = 3 | 0.210 | 9.62e−11 |
| to 14, preferably 3 to 10, more preferably 5-7, yet more | | |
| preferably y = 6) | | |
| GranzymeA+ % of CD8+ MAIT cells (d 1) | 0.126 | 7.04e−08 |
| MAIT % of CD8+ (d 0:dy, where y = 3 to 14, preferably | 0.106 | 0.00018 |
| 3 to 10, more preferably 5-7, yet more preferably y = 6) | | |
| IFNγ+ % of CD8+ MAIT cells (d 1) | 0.103 | 7.33e−05 |
| IL-17+IFNγ+ % of CD4+CD45RO+CCR6+CD161+ (dy, | 0.086 | 0.011 |
| where y = 3 to 14, preferably 3 to 10, more preferably 5-7, | | |
| yet more preferably y = 6) | | |
| CD8+CD45RO−CCR7− % of CD8+ | 0.079 | 0.035 |
| IFNγ+ % of memory CD4+ (dy, where y = 3 to 14, preferably | 0.077 | 0.00028 |
| 3 to 10, more preferably 5-7, yet more preferably y = 6) | | |
| MAIT % of CD8+ to MAIT % of DN (d 0) | 0.072 | 0.027 |
| IL-17+IFNγ+ % of CD4+CD45RO+ CCR6+ (dy, where y = | 0.071 | 1.58e−05 |
| 3 to 14, preferably 3 to 10, more preferably 5-7, yet more p | | |
| referably y = 6) | | |
| Tregs (Foxp3+Helios+) % of naïve CD4+ (d 1) | 0.069 | 0.00086 |

*Importance score determined in an RF model derived from the 40 features of Table 3.

The method can further comprise receiving other data for each human in the population. When other data is available for input to training the machine learning algorithm, extracting a set of features from the immune system data comprises extracting a set of features from the immune system data and the other data. Examples of other data for each human in the population can include clinical symptoms, demographic information, metabolic biomarkers, microbiome biomarkers, clinical history, genetics, or a combination thereof. Examples of patient demographic information include age, race, gender, weight, and the like. Examples of patient clinical history including for example smoking, alcohol consumption, blood pressure, heart rate, drug use, and current medicines being used. The genetic information can include the presence or absence of specific genetic markers.

The method can further comprise evaluating performance of the predictive model with a test set of immune system data for a population comprising healthy humans and humans with ME/CFS. Performance of the predictive model can be evaluated by applying a test set of immune system data for a population comprising healthy humans and humans with ME/CFS to determine at least one performance metric. Performance metrics of the predictive model that can be determined for the test data include sensitivity, specificity, accuracy, positive predictive value, negative predictive value, and F₁ score. Sensitivity is the proportion of true positives (ME/CFS patients) that are correctly identified by the test. Specificity is the proportion of true negatives (healthy subjects) that are correctly identified by the test. Accuracy is the proportion of the times which the classifier is correct. Positive (negative) predictive values are the proportion of positives (negatives) that are correctly identified as positives (negatives). The F₁ score measures the accuracy of the test by calculating the harmonic mean of the sensitivity and the positive predictive value.

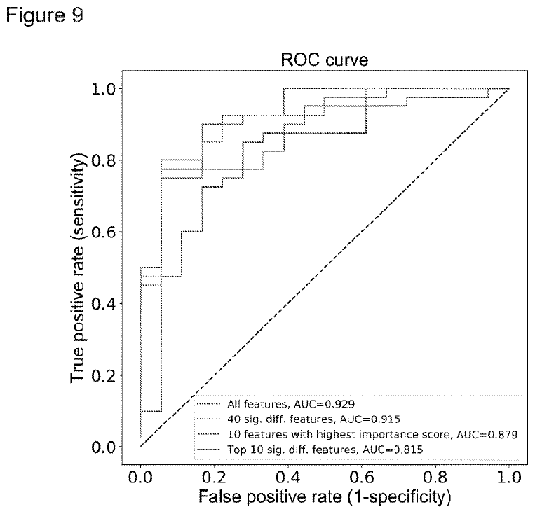

A receiver operator characteristic (ROC) curve is another possible way to evaluate performance of a predictive model. A ROC curve is created by plotting the true positive rate (TPR) against the false positive rate (FPR). For example, FIG. 9 is a graph showing ROC curves for several predictive models developed by the inventors. The dotted diagonal line in FIG. 9 is reflective of random classification. Any curves which are plotted above that line are performing better than random classification. Interpretation of ROC curves can be facilitated by calculating the area under the curve (AUC) to give a single value which explains the probability that a random subject would be correctly classified by the predictive model. An AUC of 1 represents 100% sensitivity (no false negatives) and 100% specificity (no false positives).

The sensitivity of the predictive model can be at least 0.75, at least 0.80, at least 0.82, at least 0.85, at least 0.87, at least 0.90, at least 0.91, at least 0.92, at least 0.93, at least 0.94, at least 0.95, at least 0.96, at least 0.97, or at least 0.98. The specificity of the predictive model can be at least 0.65, at least 0.70, at least 0.72, at least 0.75, at least 0.77, at least 0.80, at least 0.82, at least 0.85, at least 0.87, at least 0.90, at least 0.91, at least 0.92, at least 0.93, at least 0.94, at least 0.95, at least 0.96, at least 0.97, or at least 0.98. In certain embodiments, the F₁ score of the predictive model can be at least 0.75, at least 0.80, at least 0.82, at least 0.85, at least 0.87, at least 0.90, at least 0.91, at least 0.92, at least 0.93, at least 0.94, at least 0.95, at least 0.96, at least 0.97, or at least 0.98. The positive predictive value of the predictive model can be at least 0.75, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, or at least 0.98. The negative predictive value of the predictive model can be at least 0.55, at least 0.60, at least 0.65, at least 0.70, at least 0.75, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, or at least 0.98. The accuracy of the predictive model can be at least 0.70, at least 0.75, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, or at least 0.98. The AUC of the predictive model can be at least 0.75, at least 0.80, at least 0.82, at least 0.85, at least 0.87, at least 0.90, at least 0.91, at least 0.92, at least 0.93, at least 0.94, at least 0.95, at least 0.96, at least 0.97, or at least 0.98.

Performance of the predictive model can be evaluated using sensitivity, specificity, accuracy, positive predictive value, negative predictive value, F₁ score, a receiver operating characteristic (ROC) curve, or a combination thereof.

A method and system for diagnosing myalgic encephalomyelitis/chronic fatigue syndrome (ME/CFS) in a subject is also disclosed. The method can comprise receiving immune system data of a subject; extracting a set of features from the immune system data; inputting the features to a machine-trained classifier, the machine trained classifier trained, at least in part, from training data comprising immune system data for a population comprising healthy humans and humans with myalgic encephalomyelitis/chronic fatigue syndrome (ME/CFS); classifying, by application of the machine-trained classifier to the features, the subject as being healthy or having ME/CFS; and outputting the classification.

The system comprises a processor; and a memory storing computer executable instructions, which when executed by the processor cause the processor to perform operations comprising: comprises: receiving immune system data of a subject; extracting a set of features from the immune system data; inputting the features to a machine-trained classifier, the machine trained classifier trained, at least in part, from training data comprising immune system data for a population comprising healthy humans and humans with ME/CFS; classifying, by application of the machine-trained classifier to the features, the subject as being healthy or having ME/CFS; and outputting the classification.

The immune system data obtained for the subject can be obtained by any suitable method, such as those discussed above.

The immune system data obtained for the subject can be data for at least one of the features listed in Table 2, data for at least the ten features listed in Table 4, data for at least the first ten features listed in Table 3, data for all of the features listed in Table 3, or data for all of the features listed in Table 2.

The method can further comprise receiving other data for the subject. The other data for the subject can comprise symptoms, demographic information, metabolic biomarkers, clinical history, genetics, or a combination thereof. Extracting a set of features from the immune system data can comprise extracting a set of features from the immune system data and the other data.

The method can further comprise treating a subject classified as having ME/CFS with activity management, a prescription sleep medicine, a pain relieving drug, a pain management method, an antidepressant, an anti-anxiety drug, a stress management method, or a combination thereof.

The systems and methods described herein may be implemented in hardware, software (e.g., firmware), or a combination thereof. In some embodiments, the methods described may be implemented, at least in part, in hardware and may be part of the microprocessor of a special or general-purpose computer system, such as a personal computer, workstation, minicomputer, or mainframe computer.

In some embodiments, the computer system includes a processor, memory coupled to a memory controller, and one or more input devices and/or output devices, such as peripherals, that are communicatively coupled via a local I/O controller. These devices may include, for example, a printer, a scanner, a microphone, and the like. Input devices such as a conventional keyboard and mouse may be coupled to the I/O controller. The I/O controller may be, for example, one or more buses or other wired or wireless connections, as are known in the art. The I/O controller may have additional elements to enable communications.

Systems and methods according to this disclosure may be embodied, in whole or in part, in computer program products or in computer systems.

Technical effects and benefits of some embodiments include permitting classification of a subject as a ME/CFS patient based on the subject's immune profile to improve diagnosis and treatment of this clinical problem.

The following example is merely illustrative of the methods and systems disclosed herein and is not intended to limit the scope hereof.

### EXAMPLE

### Materials and Methods

### Participants

All subjects were recruited at Bateman Horne Center, Salt Lake City, Utah, based on who met the 1994 CDC Fukuda (Fukuda et al., 1994, Ann Intern Med 121, 953-959. 10.7326/0003-4819-121-12-199412150-00009) and/or Canadian consensus criteria for ME/CFS (Carruthers, 2007, J Clin Pathol 60, 117-119. 10.1136/jcp.2006.042754). Healthy controls were frequency-matched to cases on age, sex, race/ethnicity, geographic/clinical site and season of sampling. Patients or controls taking antibiotics, having had any infections in the prior months, or taking any immunomodulatory medications were excluded from the study. The study was approved by Western IRB (Protocol number 20151965) and written informed consent and verbal assent when appropriate were obtained from all participants in this study. We enrolled a total of 198 ME/CFS patients and 91 healthy controls. Subject characteristics are shown in Table 1.

TABLE 1
Characteristics of study subjects

| | | ME/CFS | Healthy |
|---|---|---|---|
| Demographics | | (n = 198) | Controls (n = 91) |
| Sex | Female | 151 | 63 |
| | Male | 47 | 28 |
| Age | Mean (+−SEM) | 45.92 (+−1.08) | 39.92 (+−1.60) |
| | Median | 46.5 | 38 |
| | Younger than 50 | 107 | 58 |
| | 50 or Older | 91 | 33 |

### PBMC Isolation and Preservation

Healthy and patient blood samples are obtained from Bateman Home Center, Salt Lake City, Utah and approved by Western IRB. Heparinized blood samples were shipped overnight at room temperature. Peripheral blood mononuclear cells (PBMC) were then isolated using FICOLL-PAQUE PLUS density gradient media (a sterile, ready-to-use density media containing Ficoll PM400, sodium diatrizoate, and disodium calcium EDTA; GE Healthcare), and cryopreserved in liquid nitrogen.

### Cell Surface and Intracellular Staining and Flow Cytometry Analysis

After thawing, PBMC were counted and divided into 2 parts, 1 part for day 0 surface staining, and the other part was cultured in complete RPMI 1640 medium (RPMI plus 10% Fetal Bovine Serum (FBS) (Atlanta Biologicals), and 1% penicillin/streptomycin (Corning Cellgro) supplemented with IL-2+IL15 (20 ng/ml) for Treg subsets day 1 surface and transcription factors staining, IL-7 (20 ng/ml) for day 1 and day 6 intracellular cytokine staining and a combination of cytokines (20 ng/ml IL-12, 20 ng/ml IL-15, and 40 ng/ml IL-18) for day 1 intracellular cytokine staining (IL-12 from R&D, IL-7 and IL-15 from Biolegend).

Surface staining was performed in staining buffer containing PBS (Phosphate buffer Saline)+2% FBS for 30 minutes at 4° C. When staining for chemokine receptors the incubation was done at room temperature. Antibodies used in the surface staining were CD3 (UCHT1 clone, Alexa Fluor 532, eBIOSCIENCE), CD4 (OKT4 clone, Brilliant Violet 510), CD8 (RPA-T8 clone, Pacific Blue or Brilliant Violet 570), CD19 (HIB19 clone, Brilliant Violet 510), CD45RO (UCHL1 clone, Brilliant Violet 711, APC/Cy7, or Brilliant Violet 570), CCR7 (G043H7 clone, Alexa Fluor 488), 2B4 (C1.7 clone, PerCP/Cy5.5), CD14 (HCD14 clone, Alexa Fluor 700), CD27 (0323 clone, PE/Cy7, Brilliant Violet 605, or Alexa Fluor 647), CCR6 (G034E3 clone, Brilliant Violet 605), CD161 (HP-3G10, Brilliant Violet 421), Va7.2 (3C10 clone, PE) (all from Biolegend).

For intracellular cytokine staining, cells were stimulated with phorbol 12-myristate-13-acetate (PMA; 40 ng/ml for overnight cultured cells and 20 ng/ml for 6 days cultured cells) and ionomycin (500 ng/ml) (both from Sigma-Aldrich) in the presence of GOLGISTOP (a protein transport inhibitor containing monensin, BD Biosciences) for 4 hours at 37° C. For cytokine secretion after stimulation with IL-12+IL-15+IL-18+, GOLGISTOP was added to the culture on day 1 for 4 hours. Stimulated or unstimulated cells were collected, stained with surface markers including CD3, CD4, CD8, CD161, Vα7.2, CD45RO, CCR6, and CD27 (all from Biolegend) followed by one wash with PBS (Phosphate buffer Saline) and staining with Fixable Viability Dye eFLUOR™ 780 (eBIOSCIENCE™ Cat #65-0865-14). After surface staining, cells were fixed and permeabilized using Intracellular Fixation & Permeabilization Buffer Set (eBIOSCIENCE™) according to the manufacturer's instruction. Permeabilized cells were then stained for intracellular IFNγ (4S.B3 clone, APC/Cy7), TNFα (Mab11 clone, PE/Dazze 594), Granzyme A (CB9 clone, Alexa Fluor 647, Alexa Fluor 488), IL-17A (BL168 clone, Alexa Fluor 488, Brilliant Violet 421), Foxp3 (259D clone, PE), and Helios (22F6 clone, Alexa Fluor 488) (all from Biolegend).

Permeabilized cells were then stained for intracellular IFNγ (4S.B3 clone, APC/Cy7), TNFα (Mab11 clone, PE/Dazze 594), GranzymeA (CB9 clone, Alexa Fluor 647, Alexa Fluor 488), IL-17A (BL168 clone, Alexa Fluor 488, Brilliant Violet 421), Foxp3 (259D clone, PE), and Helios (22F6 clone, Alexa Fluor 488) (all from Biolegend).

Flow cytometry analysis was performed using a SP6800 Spectral Cell Analyzer (Sony Biotechnology) and analyzed using FlowJo version 10 (Tree Star).

### Machine Learning and Statistical Analysis

All statistical analyses were performed using GraphPad Prism V8 software. Continuous variable datasets were analyzed by Mann-Whitney U test for non-parametric datasets when comparing clinical groups, and exact P values are reported. Spearman p was used to determine the relationship existing between two sets of data between non-parametric datasets.

The algorithms for identifying significantly different features and the RF classifier were implemented in Python 3.6.8 using Jupyter Notebook 5.0.0. The RF classifier was performed with different numbers of features of k=65, 40, and 10. A training set with 231 samples (80% of total samples) was selected and the remaining data corresponding to 58 samples (20% of total samples) was left as the test set. Missing values in the training and test sets were replaced by the corresponding median value in the training set. The RF classifier was implemented using a 3-fold (stratified) cross validation and was trained using all 65 immune profile features, the 40 significantly different features, the top 10 significantly different features and the top 10 features among the 40 significantly different features that received the highest importance score.

There are several metrics to evaluate the performance of a classifier. Sensitivity represents the proportion of patients who were correctly identified as patients and specificity represents the proportion of healthy controls who were correctly identified as healthy. If patients are denoted by “positives” and healthy controls by “negatives”, then sensitivity and specificity are calculated as:

$$sensitivity = \frac{True Positives}{Positives} and specificity = \frac{True Negatives}{Negatives}$$

where “true positives” refer to patients who were correctly identified as patients and “true negatives” refer to healthy controls who were correctly identified as healthy.

Accuracy is a metric which shows the fraction of predictions that our classifier predicted correctly. Accuracy is calculated in terms of true positives and true negatives as follows:

$$Accuracy = \frac{True positives + True negatives}{Total number of predictions}$$

Positive (negative) predictive values are the proportion of positives (negatives) that are correctly identified as positives (negatives) which are calculated as follows:

$$Positive predictive value = \frac{True positives}{True positives + False positives} \\ Negative predictive value = \frac{True negatives}{True negatives + False negatives}$$

The F₁ score measures the accuracy of test by calculating the (harmonic) mean of the sensitivity (recall) and positive predictive value (precision). The F₁ score is defined as:

$${F}_{1} = 2 \times \frac{precision \times recall}{precision + recall}$$

### Results

### Changes in T Cells Subsets in ME/CFS Patient Blood

To determine phenotypic and functional changes in immune cell subsets from ME/CFS patients, we developed several flow cytometry staining panels and performed high resolution immune profiling of 198 ME/CFS patients and 91 age- and sex-matched healthy controls (Table 1).

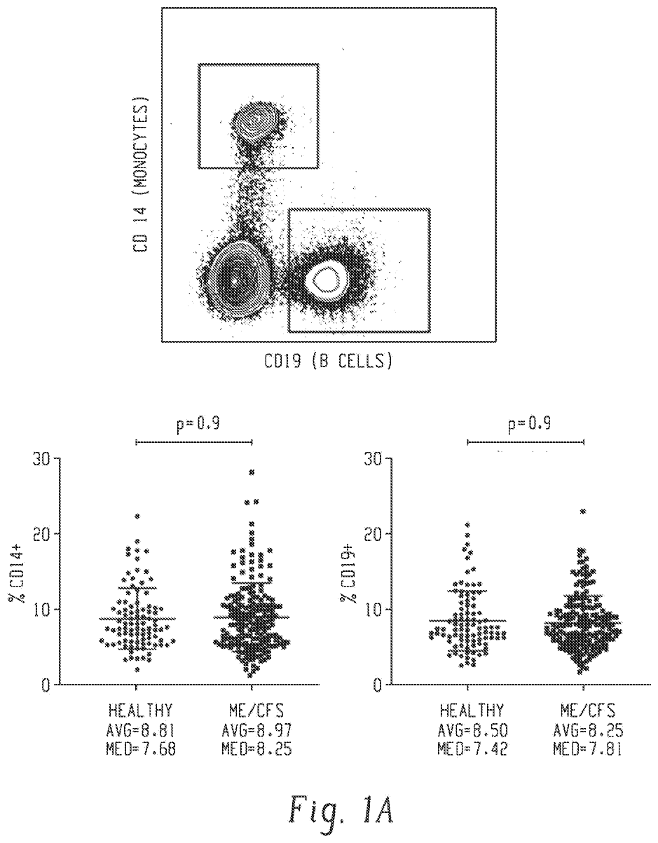

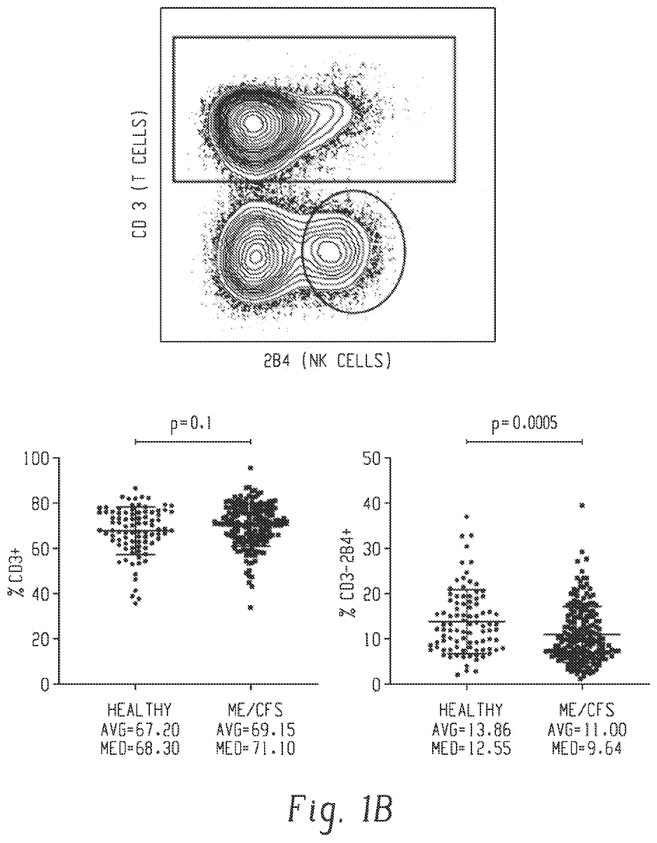

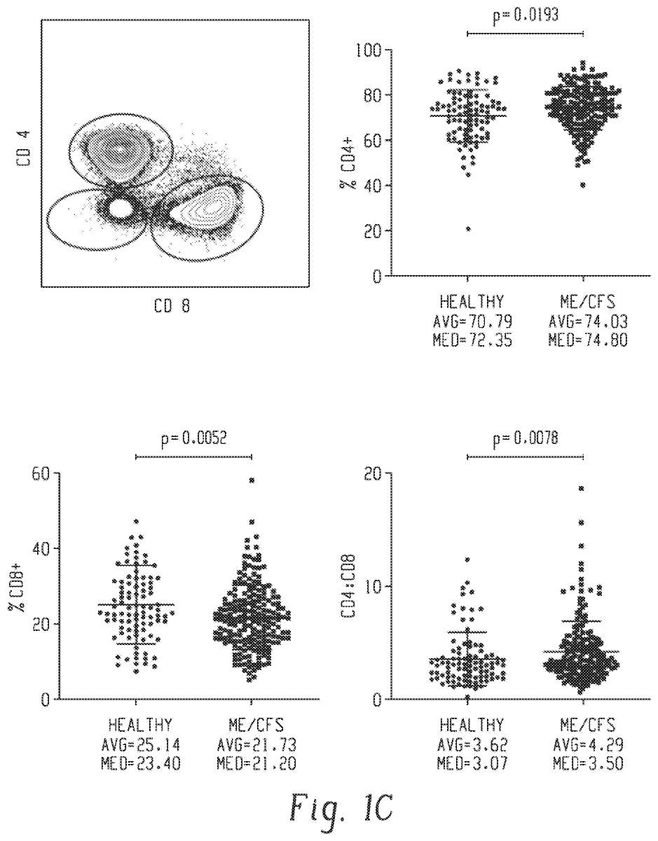

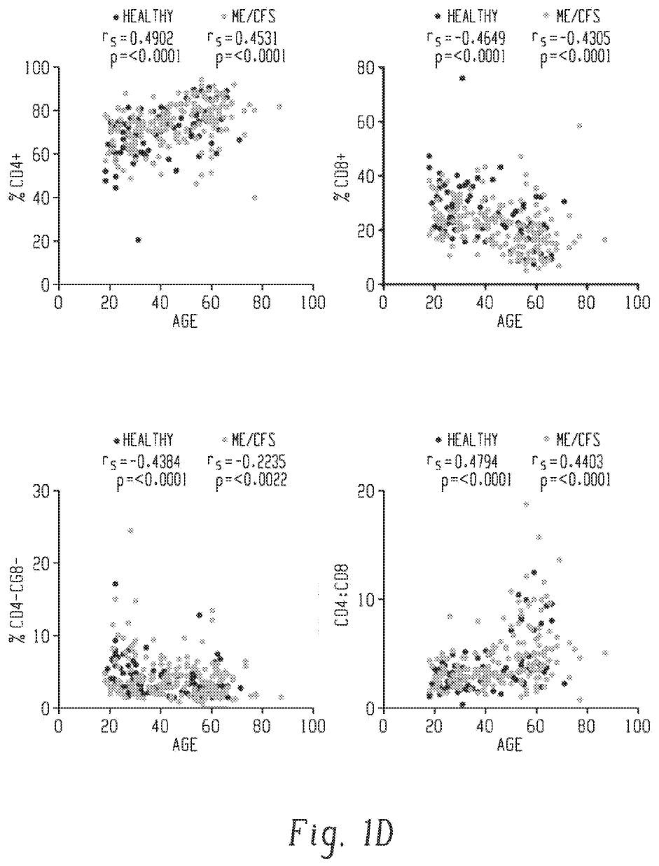

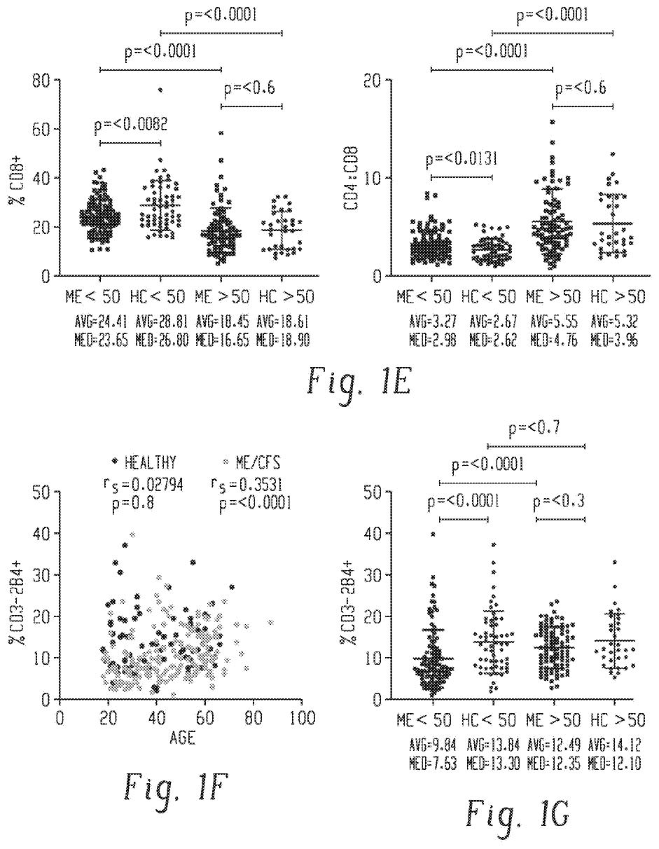

We first analyzed the main immune subsets in peripheral blood mononuclear cells (PBMCs), namely T cells, B cells, NK cells, and monocytes (FIG. 1a, 1b). There was no significant difference in the percentage of overall monocytes (p=0.9), B cells (p=0.9) or T cells (p=0.1) (FIG. 1a, 1b), but the frequency of NK cells within lymphocytes was greatly reduced (p=0.0005) in ME/CFS compared to healthy controls (FIG. 1b). Within T cells, we observed that CD4+ T cell frequency was higher (p=0.0193) and correspondingly, CD8+ T cells were lower (p=0.0052) in ME/CFS subjects, and that this was reflected as a higher CD4 to CD8 ratio (p=0.0078) in patients (FIG. 1c). There was no difference in CD4− CD8− (double negative; DN) T cells (p=0.9) between controls and ME/CFS patients (data not shown).

Changes in the CD4 to CD8 ratio are associated with normal aging (Yan, J. et al. Immun Ageing 7, 4, doi:10.1186/1742-4933-7-4 (2010); Serrano-Villar, S. et al. HIV Med 15, 40-49, doi:10.1111/hiv.12081 (2014)). Indeed, CD4+ and CD8+ T cell frequencies and the CD4 to CD8 ratio correlated with age in both healthy controls (rₛ=0.4902, −0.4649, and 0.4794 respectively) and in ME/CFS subjects (rₛ=0.4531, −0.4305, and 0.4403, respectively) (FIG. 1d). Age also showed a significant correlation with DN T cells for healthy controls (rₛ=−0.4384), but not for ME/CFS patients (rₛ=−0.2235) (FIG. 1d). Interestingly, when we subdivided ME/CFS patients and healthy controls into two groups consisting of subjects who were younger than 50 years of age and subjects who were 50 years of age or older, the difference in percentages of CD8+ T cells and the CD4:CD8 ratio remained significant for subjects younger than 50 (p=0.0082 and 0.0131, respectively), but was not significant for subjects 50 and older (p=0.6 and 0.6, respectively). (FIG. 1e). Age also showed a significant correlation with NK cell frequency only in ME/CFS patients (rₛ=0.3531), but not in healthy controls (rₛ=0.02794) (FIG. 1f). This change in NK cell frequency was also only seen in ME/CFS subjects younger than 50 years (p<0.0001) (FIG. 1g).

We next divided CD4+ and CD8+ T cells into naïve and memory subsets as part of their differentiation states, based on their functional and phenotypic features (Sallusto et al., 2004, Central memory and effector memory T cell subsets: function, generation, and maintenance. Annu Rev Immunol 22, 745-763. 10.1146/annurev.immunol.22.012703.104702). To determine the proportion of these subsets in ME/CFS patients, we used well-established CD45RO and CCR7 cell surface molecules as markers for both CD4+ and CD8+ T cell subsets (FIG. 2a). Within CD4+ T cells, there was no significant difference between ME/CFS patients and healthy controls for CD45RO-CCR7+ (naïve; N) (p=0.5), CD45RO+CCR7+ (central memory; CM) (p=0.7), CD45RO+CCR7− (effector memory; EM) (p=0.2), or CD45RO-CCR7− (effector memory RA; EMRA) (p=0.06) subsets (FIG. 2b). There was also no difference in CD8+ N (p=0.4), CM (p=0.1), or EMRA (0.0509) populations, however, the CD8+ EM T cell subset was greatly increased as a proportion of CD8+ T cells (p=0.0001) in ME/CFS patients (FIG. 2c).

The frequencies of N, CM, EM, and EMRA populations within CD8+ T cells correlated with age for both healthy controls (rₛ=−0.5259, 0.5222, 0.3696, and 0.3602 respectively) and ME/CFS patients (rₛ=−0.6162, 0.3756, 0.3814, and 0.5172 respectively) (FIG. 2d), and this was also seen in CD4+ subsets. However, there was no significant difference between ME/CFS patients and healthy controls for CD8+ N or CM T cells for subjects who were younger than or 50 years and older (p=0.8 and 0.07, respectively) (FIG. 2d) or for CD4+ N, CM or EM subsets in different age groups of patients and controls. Interestingly, the CD8+ EM subset difference between ME/CFS patients and healthy controls was restricted to subjects younger than 50 years (p=0.0027) (FIG. 2e). CD8+ and CD4+ EMRA subsets were also only significantly lower in ME/CFS patients who were younger than 50 years of age (p=<0.0001 and p<0.0156 respectively) (FIG. 2e).

### Changes in Th17 Cell Frequency and Function in ME/CFS Disease

We hypothesized that ME/CFS patients may also have disruptions within effector T cell subsets resident in mucosal tissues such as Th17 cells, which respond to bacterial infections or microbiota and are also linked to autoimmune diseases (Milner et al., 2010, Th17 cells, Job's syndrome and HIV: opportunities for bacterial and fungal infections. Curr Opin HIV AIDS 5, 179-183. 10.1097/COH.0b013e328335ed3e; Pandiyan et al., 2019, Microbiome Dependent Regulation of Tregs and Th17 Cells in Mucosa. Front Immunol 10, 426. 10.3389/fimmu.2019.00426). To identify Th17 cells we first used CD3, CD4, CD45RO, and CCR6 expression (FIG. 3a), as almost all of this subset has a memory phenotype and also expresses the chemokine receptor CCR6 (Romagnani et al., 2009, Properties and origin of human Th17 cells. Mol Immunol 47, 3-7. 10.1016/j.molimm 2008.12.019). In order to analyze the cytokine secretion from T cells, we thawed frozen aliquots of PBMC and cultured one day in IL-7 (d1) to ensure cells recovered from thawing and any dying cells could be clearly identified. We then activated the cells with PMA and lonomycin as described in methods. The cells were then stained intracellularly for IL-17 and IFNγ and gated on cell surface expression based on CD4+CD45RO+CCR6+ and CD4+CD45RO+CCR6− cells (FIG. 3b), as previously described (Wan et al., 2011, Cytokine signals through PI-3 kinase pathway modulate Th17 cytokine production by CCR6+ human memory T cells. J Exp Med 208, 1875-1887. 10.1084/jem.20102516). Within the CD4+CD45RO+ (memory T cell) population, the frequency of IL-17+ (p=0.0378), IFNγ+ (p=0.0231), and IL-17+IFNγ+ (p=0.0378) secreting cells was significantly reduced in ME/CFS compared to healthy subjects (FIG. 3c).

Previously we have shown that a portion of Th17 cells are poised to produce IL-17 or IL-22 only after priming with γc-cytokines (namely IL-2, IL-15, or IL-7) in culture, which reveal their full potential of their IL-17 secretion (Wan et al., 2011). Accordingly, we cultured PBMC from ME/CFS patients and control subjects for 6 days (d6) in IL-7 to prime Th17 cells for IL-17 secretion, as previously described (Wan et al., 2011). PBMC were then stimulated using PMA and lonomycin, and expression of cytokines within T cell subsets was determined. In this assay, T cells from ME/CFS patients expressed profoundly lower total IL-17+ (p<0.0001), IFNγ (p<0.0001), IL-17+IFNγ+ (p<0.0001), and IL-17+IFNγ− (p<0.0001) cells compared to healthy controls (FIG. 3d), revealing a major dysfunction of Th17 cells in patients.

After 6 days in culture with IL-7, the proportion of IL-17 and IFNγ secreting cells within CD4+CD45RO+ memory population of healthy controls did not correlate with age for either IL-17+, IFNγ+, IL-17+IFNγ+, or IL-17+IFNγ−subsets (rₛ=−0.2379, −0.2929, −0.2413, and −0.2719 respectively) (FIG. 3e). For ME/CFS patients, age also did not correlate with IFNγ+ expressing cells (rₛ=−0.2929), but IL-17+, IL-17+IFNγ+, and IL-17+IFNγ− subsets showed a significant correlation (rₛ=−0.4073, −0.3952, and −0.3784, respectively) (FIG. 3e). When patients were broken down into groups of subjects younger than and >50 years, significant differences were observed in IL-17+, IFNγ+, IL-17+IFNγ+, and IL-17+IFNγ− subsets between controls and ME/CFS subjects younger than 50 years (p=0.0017, 0.0018, 0.0009, and 0.0009, respectively), as well as among the >50 years groups (p=0.0002, 0.0047, 0.0008, and 0.0001, respectively) (FIG. 3f).

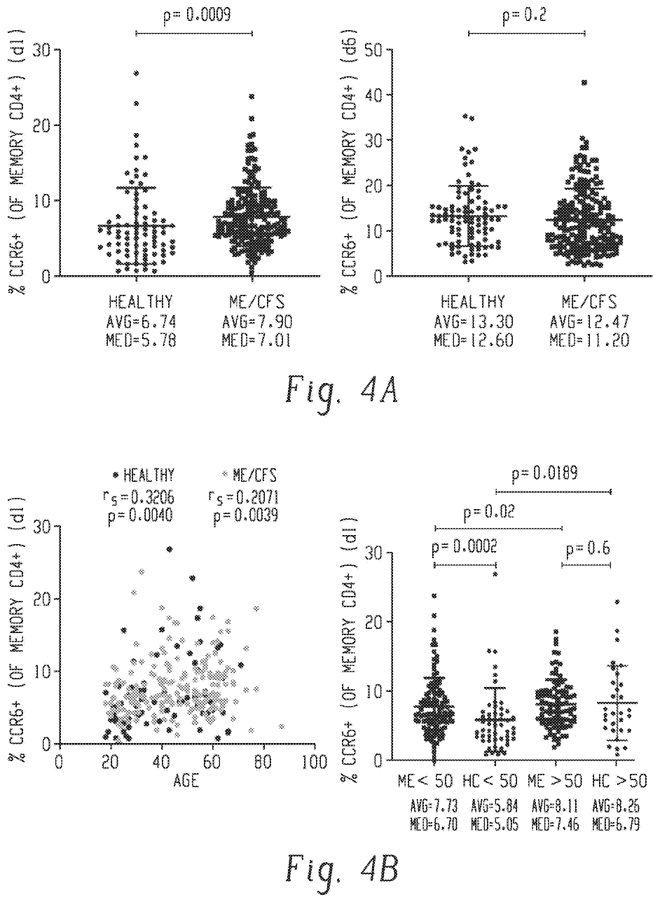

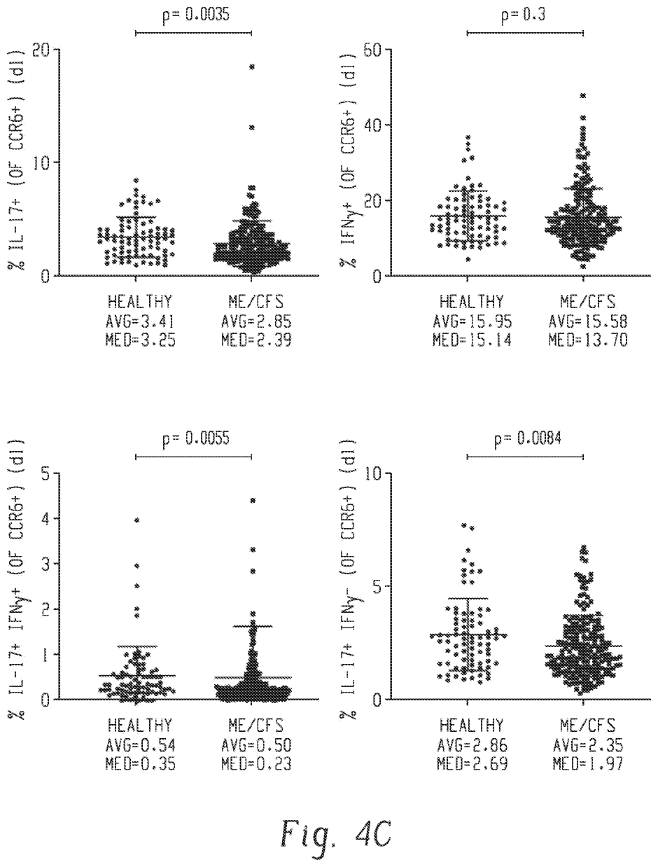

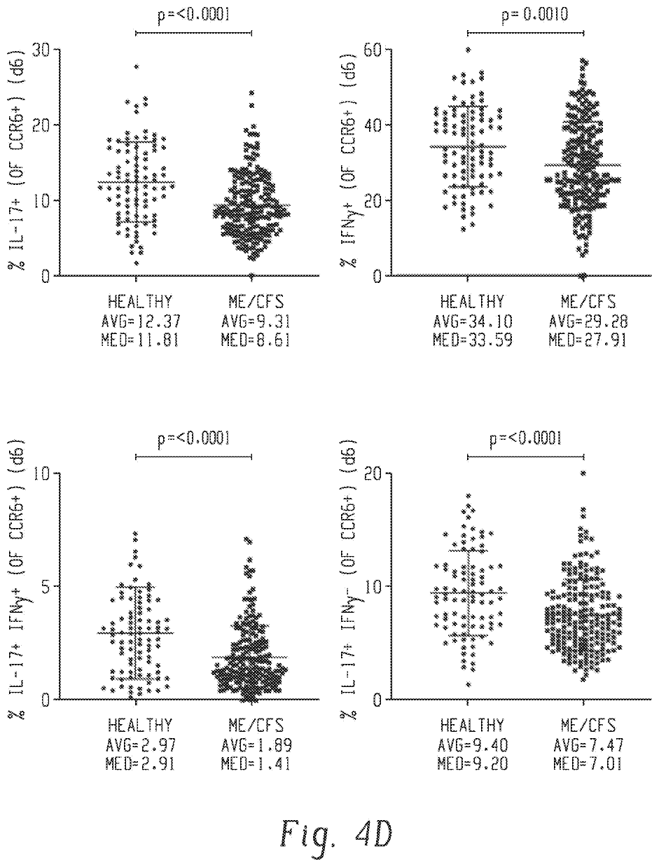

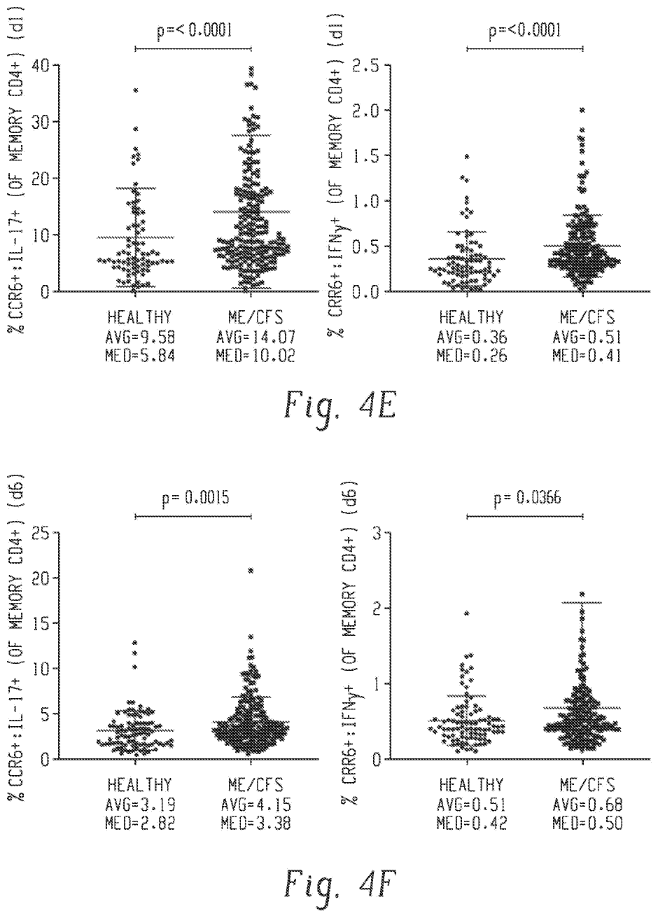

To further investigate the disruption in the Th17 cell subset, we compared the frequency of CD4+CD45RO+CCR6+ cells between controls and ME/CFS patients. In contrast to IL-17 expression, we found that CCR6+ cells were significantly higher in ME/CFS patients after 1 day in culture in IL-7 (p=0.0009) (FIG. 4a). However, after 6 days in IL-7, there was no difference between the subject groups (p=0.2), even though the average frequency was higher in both groups (FIG. 4a). CCR6+ cell frequency within memory CD4 T cells correlated with subject age for healthy controls (rₛ=0.3206), but not for ME/CFS patients (rₛ=0.2071). When patients were grouped as younger than and >50 years of age, a significant difference was seen for the proportion of CCR6+ cells only in subjects younger than 50 years (p=0.0002) (FIG. 4b).

Remarkably, ME/CFS subjects, compared to controls, displayed lower expression of IL-17+ (p=0.0035), IL-17+IFNγ+ (p=0.0055), and IL-17+IFNγ−(p=0.0084), but not total IFNγ+ (p=0.3), within the CD4+CD45RO+CCR6+ T cells (FIG. 4c). After 6 days in culture in IL-7, the differences further increased and were seen in all cytokine-secreting cells, as a proportion of CD4+CD45RO+CCR6+ T cells, for IL-17+ (p<0.0001), IFNγ+ (p=0.0010), IL-17+IFNγ+ (p<0.0001), and IL-17+IFNγ−(p<0.0001) cells (FIG. 4d).

We next determined the ratio between the CCR6+ T cells to CD4+ memory T cells expressing IL-17 or IFNγ. Indeed, the ratio of CCR6+ cells to IL-17+ (p<0.0001) and to IFNγ+ (p<0.0001) CD4+ memory T cells were significant in ME/CFS patients compared to healthy controls (FIG. 4e). These ratios between CCR6+ cells and cytokines produced by CD4+ cells also remained higher in ME/CFS subjects after d6 in IL-7, for CCR6+ to IL-17+ cell ratio (p=0.0015), but were only marginally different for CCR6+ to IFNγ+ cell ratio (p=0.0366) (FIG. 4f).

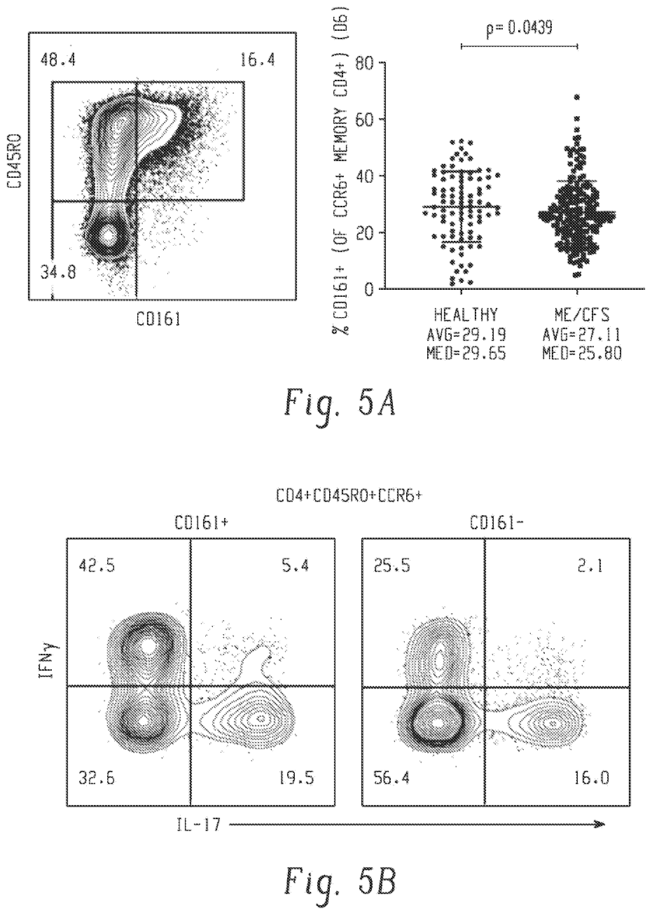

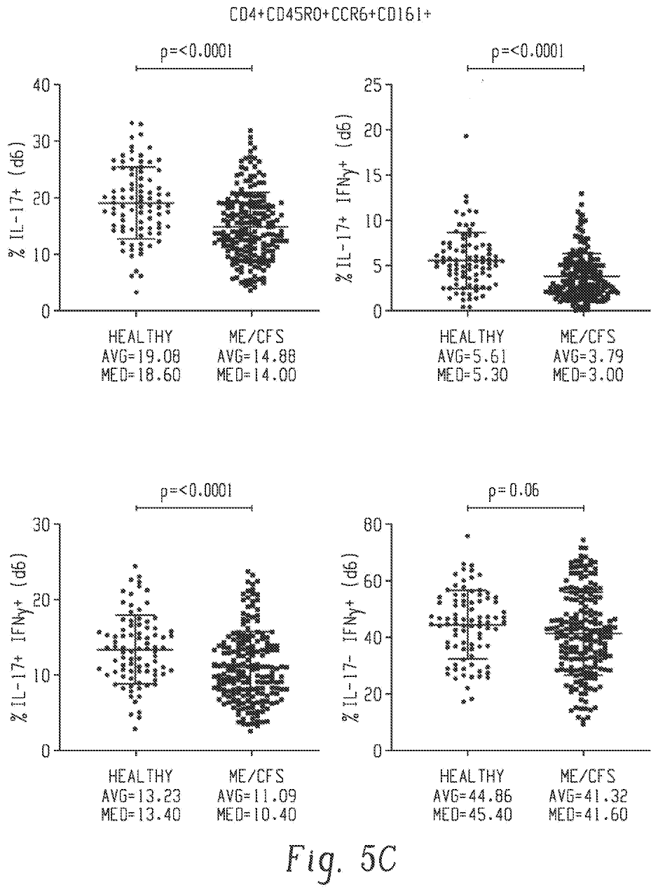

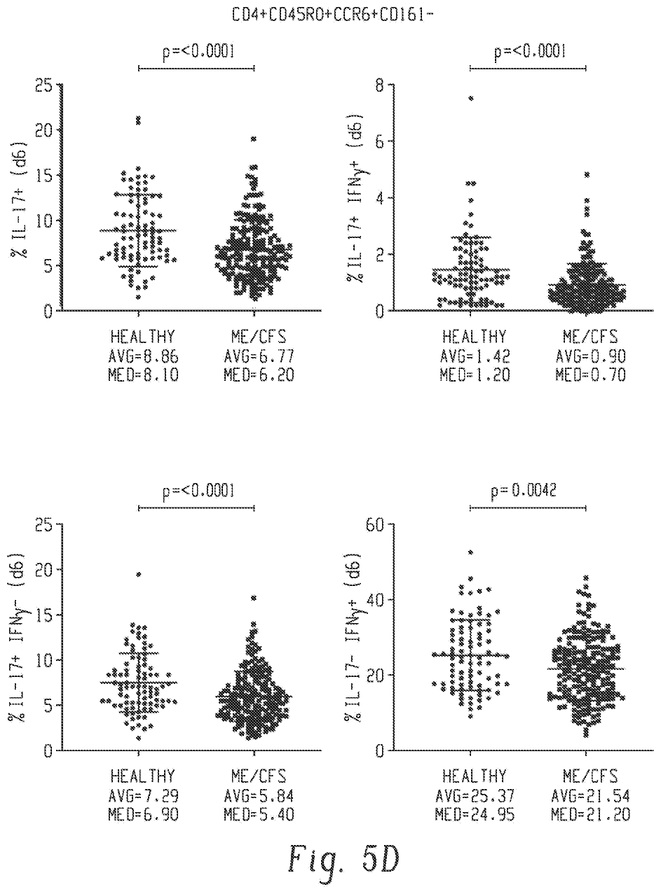

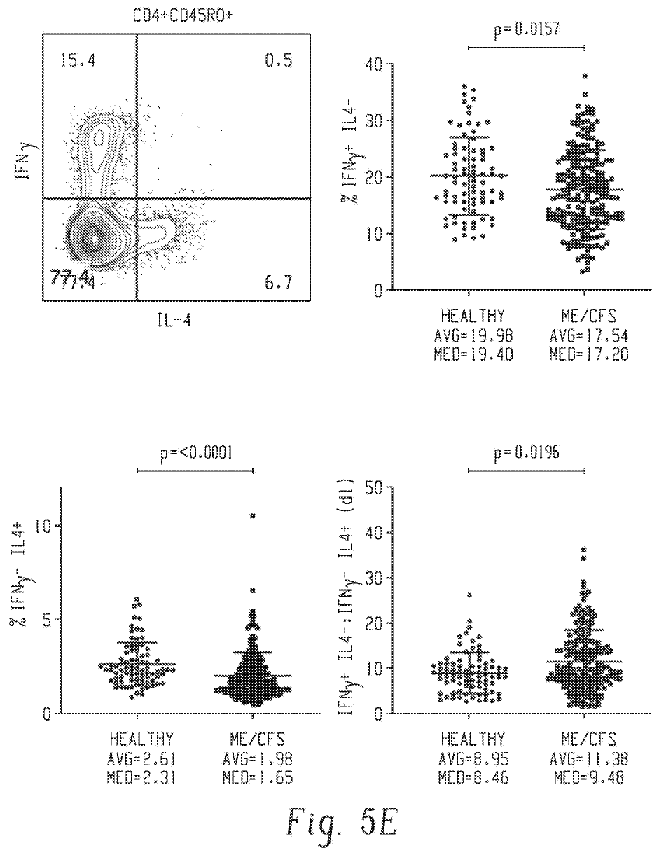

We have previously shown that CD161 within the CD4+CD45RO+CCR6+ T cells can further divide these cells into subsets with differences in IL-17 and IFNγ secretion (Wan et al., 2011). As such, we further divided CCR6+ cells based on CD161 expression (FIG. 5a). The proportion of CD161+ cells within the CCR6+ subset was only slightly different in ME/CFS compared to controls (p=0.0439) (FIG. 5a). We then analyzed IL-17 and IFNγ expression within the CD161+ and CD161− subsets of CD4+CD45RO+CCR6+ cells after 6 days in culture (FIG. 5b). Within the CD4+CCR6+CD161+ cells, there was a significant difference in expression of IL-17+, IL-17+IFNγ+, and IL-17+IFNγ− cells between ME/CFS and controls (p<0.0001 for all), but not for IL-17-IFNγ+ cells (p=0.06) (FIG. 5c). CD161− cells within the CD4+CD45RO+CCR6+ subset also displayed lower IL-17+, IL-17+IFNγ+, IL-17+IFNγ−, and IL-17-IFNγ+ in ME/CFS subjects compared to healthy controls (p=<0.0001, <0.0001, 0.0001, and 0.0042, respectively), (FIG. 5d).

In CD4+ memory T cells, in addition to IL-17 expression, we also determined the frequency of T cells that were either expressing IFNγ (IFNγ+IL-4-) or IL-4 (IFNγ-IL-4+) only, which respectively define Th1 and Th2 T cell subsets (FIG. 5e). We found that the proportion of IFNγ+IL-4− and IFNγ-IL-4+ within CD4+ memory T cells was significantly lower (p=0.0157 and p<0.0001 respectively) in ME/CFS subjects (FIG. 5e). However, the ratio of Th1 (IFNγ+IL-4−) to Th2 (IFNγ-IL-4+) was higher in ME/CFS patients compared to the control group (p=0.0196) (FIG. 5e), suggesting an imbalance of Th1 to Th2 cells. Together, these findings highlight major functional perturbations within the CD4+ T cell subset in the ME/CFS patient cohort.

### Changes in Frequency of MAIT Cells in ME/CFS

Mucosal-associated invariant T (MAIT) cells are a subset of the non-classical T cell population and defined by an invariant T cell receptor that is triggered by riboflavin metabolites produced by bacteria, including commensal microbiota (Tastan et al., 2018, Tuning of human MAIT cell activation by commensal bacteria species and MR1-dependent T-cell presentation. Mucosal Immunol 11, 1591-1605. 10.1038/s41385-018-0072-x; Godfrey et al., 2019, The biology and functional importance of MAIT cells. Nat Immunol 20, 1110-1128. 10.1038/s41590-019-0444-8). Similar to the Th17 subset, we hypothesized that dysbiosis in the gut microbiome or prior bacterial infections may result in changes in MAIT cell frequencies or function. To identify MAIT cells in PBMC, we used Vα7.2 and CD161 surface molecules as previously described (Khaitan et al., 2016, HIV-Infected Children Have Lower Frequencies of CD8+ Mucosal-Associated Invariant T (MAIT) Cells that Correlate with Innate, Th17 and Th22 Cell Subsets. PLoS One 11, e0161786. 10.1371/journal.pone.0161786; Tastan et al., 2018). We then determined the frequency of MAIT cells within CD4+, CD8+ and CD4-CD8− (double negative or DN) T cell compartments in ME/CFS patients and healthy controls (FIG. 6a). There was no significant difference between patients and controls for CD4+ (p=0.7), CD8+ (p=0.7), or double negative (DN) MAIT cells (p=0.2) as a proportion of the CD4+, CD8+ and DN T cells respectively (FIG. 6b). However, CD4+ and CD8+ MAIT cell frequencies in PBMC after 6-day culture in IL-7 showed a significant difference (p=0.0250 and p=0.0221 respectively) between ME/CFS patients and controls, but DN MAIT cell frequency did not change between ME/CFS and control samples after 6 days culture (p=0.3) (FIG. 6c). When the ratio of MAIT cell frequency at day 0 (d0) vs day 6 after IL-7 culture (d6) was assessed, we found that the frequency of CD8+ MAIT cells in ME/CFS PBMC was greatly reduced after 6 days of culture compared to d0 levels (p=0.0008), but there was no significant difference seen for CD4+ (p=0.06) or for DN MAIT cells (p=0.8) between ME/CFS patients and controls (FIG. 6d). A corollary to this finding, the ratio of CD8+ MAIT to DN MAIT cells in ME/CFS patients and controls was only slightly significant at d0 (p=0.0364), but became highly significant after 6 days in culture with IL-7 (p<0.0001) (FIG. 6e). Together, these findings suggest that CD8+ MAIT cells from ME/CFS subjects survived less in in vitro culture with IL-7.

Because CD27 expression on MAIT cells could indicate a recently activated or differentiated subset, similar to other CD8 T cells (Dolfi and Katsikis, 2007, CD28 and CD27 costimulation of CD8+ T cells: a story of survival. Adv Exp Med Biol 590, 149-170. 10.1007/978-0-387-34814-8_11; Grant et al., 2017, The role of CD27 in anti-viral T-cell immunity. Curr Opin Virol 22, 77-88. 10.1016/j.coviro.2016.12.001), we evaluated CD27 expression in MAIT subsets (FIG. 6f). We found that ME/CFS patients had a significant difference where there were higher CD45RO+CD27− cells compared to the control group (p=0.0045), but interestingly, this difference was not observed within DN MAIT cells (p=0.7) (FIG. 6g). The d0 to d6 CD8+ MAIT cell ratio also displayed a slight positive correlation with the frequency of CD27-CD8+ MAIT cells in patients (rₛ=0.2920), but not in healthy controls (rₛ=0.2519). In contrast, CD27− DN MAIT cells did not correlate with the d0 to d6 cell frequency ratio for either controls (rₛ=0.1597), or ME/CFS patients (rₛ=0.1016) (FIG. 6h).

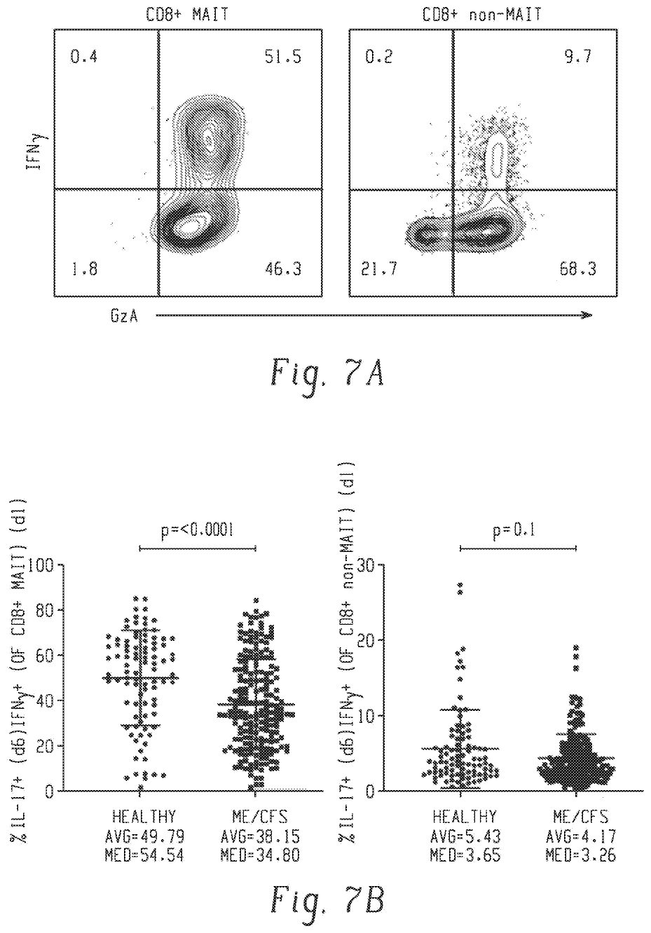

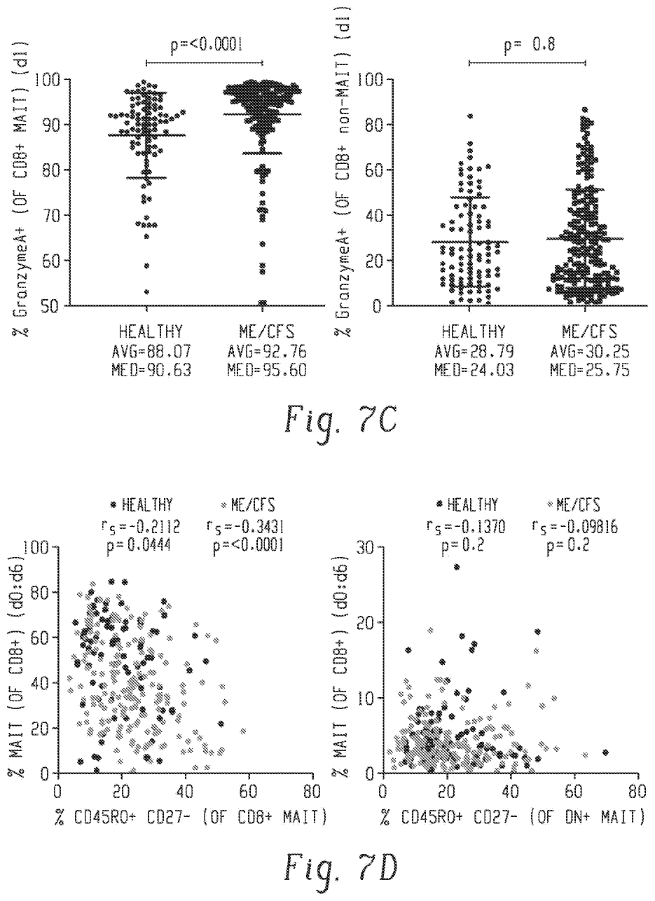

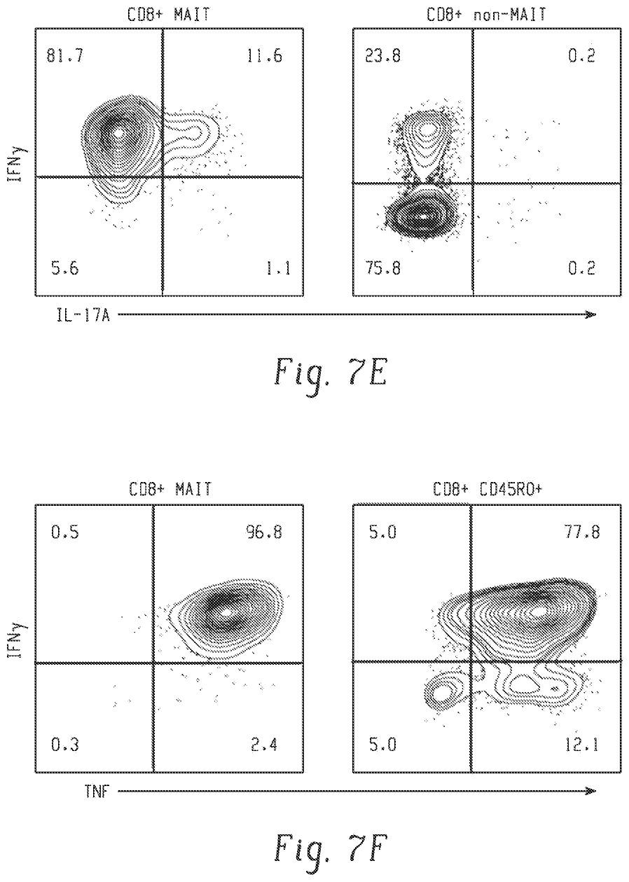

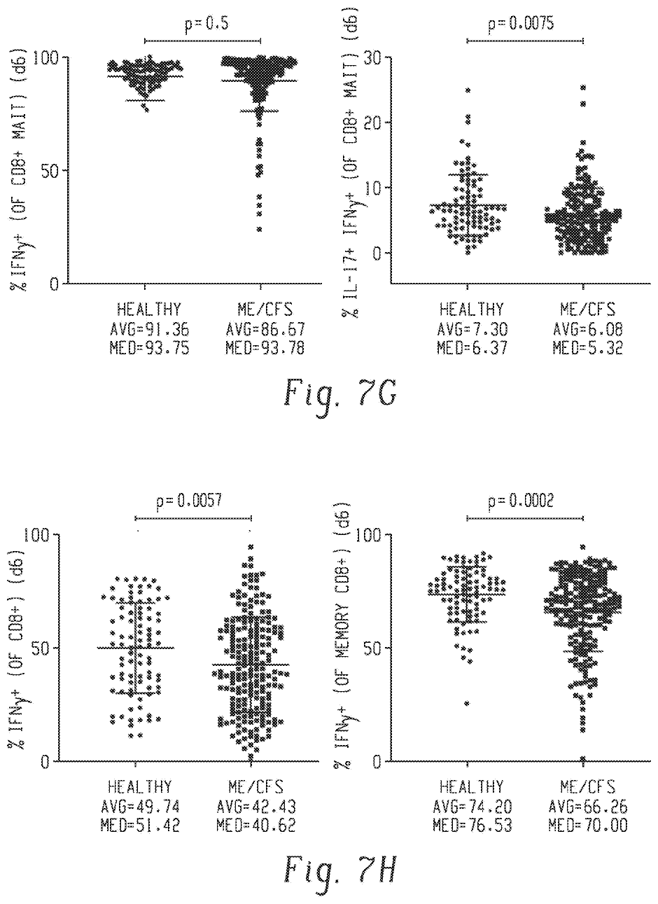

We then asked to what extent MAIT cells were functionally different between ME/CFS patients and controls. For this approach we first stimulated the PBMC with a cocktail of three cytokines, namely IL-12+IL-15+IL18, since this combination has been uniquely shown to induce expression of IFNγ from MAIT cells (Ussher et al., 2014, CD161++CD8+ T cells, including the MAIT cell subset, are specifically activated by IL-12+IL-18 in a TCR-independent manner Eur J Immunol 44, 195-203. 10.1002/eji.201343509; Salou et al., 2017, MAIT cells in infectious diseases. Curr Opin Immunol 48, 7-14. 10.1016/j.coi.2017.07.009). Accordingly, IFNγ along with Granzyme A expression was used to evaluate the response of CD8+ MAIT and CD8+ non-MAIT cells in PBMC to stimulation with IL-12+IL-15+IL18 cocktail (FIG. 7a). ME/CFS patient PBMC stimulated with the cytokine cocktail showed much lower IFNγ+ MAIT cells (p=<0.0001), but induction of IFNγ+ from non-MAIT CD8+ T cells was comparable (p=0.1) to healthy subjects (FIG. 7b). Granzyme A expressing MAIT cells were also much higher in ME/CFS subjects (p<0.0001), but non-MAIT cells expressing Granzyme A were not different (p=0.8) between controls and patients (FIG. 7c). In addition, CD27-CD8+ MAIT cells and IFNγ+ MAIT cells upon cytokine stimulation were negatively correlated in ME/CFS patients (rₛ=−0.3431; p<0.0001) but not in controls (rₛ=−0.2112; p=0.0444) (FIG. 7d). CD27-CD8+ MAIT cells were not correlated with CD8+ non-MAIT IFNγ+ cells for either healthy controls (rₛ=−0.1370; p=0.2) or ME/CFS patients (rₛ=−0.09816; p=0.2) (FIG. 7d).

Since MAIT cells have also been shown to express IL-17, similar to Th17 cells (Salou et al., 2017), we next sought to determine the production of IL-17 and IFNγ from MAIT cells in response to PMA and lonomycin stimulation. There was very little to undetectable IL-17 expression from MAIT cells after one day in culture (data not shown). However, after 6 days in IL-7, MAIT cells expressing IL-17 were greatly increased upon PMA and lonomycin stimulation, however, IL-17 remained undetectable in non-MAIT CD8+ T cells (FIG. 7e). This finding suggests that MAIT cells can also undergo priming with cytokines, similar to the classic Th17 cells (Wan et al., 2011) and IL-17 expression mimics tissue-resident MAIT cells (Sobkowiak et al., 2019, Tissue-resident MAIT cell populations in human oral mucosa exhibit an activated profile and produce IL-17. Eur J Immunol 49, 133-143. 10.1002/eji.201847759).

In addition, we also determined IFNγ and TNFα secretion from CD8+ MAIT and CD8+ non-MAIT CD45RO+ (memory) T cells after 6 days in culture with IL-7 (FIG. 7f). There was no significant difference in ME/CFS patients for IFNγ+ MAIT cells (p=0.5), but we found highly reduced IL-17+IFNγ+ MAIT cells in ME/CFS patients compared to healthy controls (p=0.0075) (FIG. 7g). The frequency of IFNγ+ secreting cells was also reduced within CD8+ non-MAIT cells (p=0.0057) and within CD8+CD45RO+ memory T cells (p=0.0002), in ME/CFS PBMC cultured for 6 days in IL-7 (FIG. 7h).

### Changes in Regulatory T (Treg) Cells in ME/CFS Patients

Regulatory T (Tregs) cells are critical in controlling autoreactive or excessive immune responses. Given the observed perturbance in the effector functions of T cell subsets that suggest chronic immune activation, we hypothesized that there would be a corresponding increase in Tregs in ME/CFS patients. For this experiment, we used Foxp3 and Helios as markers to assess Treg cell frequencies within both naïve and memory CD4+ T cells, as previously described (Mercer et al., 2014, Differentiation of IL-17-producing effector and regulatory human T cells from lineage-committed naive precursors. J Immunol 193, 1047-1054. 10.4049/jimmunol.1302936) (FIG. 8a). Indeed, frequencies of both naïve Tregs (p=0.0005), and memory Tregs (p=0.0094) were increased in ME/CFS compared to controls (FIG. 8b).

When broken down into groups where subjects were younger than or ≥50 years, naïve Tregs showed a highly significant difference in ME/CFS patients vs controls in the younger than 50 years group (p=0.0083), and a slightly significant difference in ME/CFS patients vs controls in the >50 years group (p=0.0209). The difference in memory Tregs was also significant between ME/CFS patients and controls younger than 50 years (p=0.0116), but not when the >50 years groups were compared (p=0.6) (FIG. 8c). There was no correlation with subject age for ME/CFS patients or controls for naïve Tregs (rₛ=−0.02541 and rₛ=−0.01592, respectively), or for memory Tregs (rₛ=0.2691 and rₛ=0.1585, respectively) (FIG. 8d).

The ratio of Th17 cells to Tregs is an important feature that is perturbed during chronic inflammatory conditions or autoimmune diseases. Therefore, we also determined this ratio in ME/CFS patients vs healthy controls. While the Th17 (CCR6+IL-17-secreting cells) frequency did not correlate with memory Treg cells in ME/CFS patients (rₛ=0.2750) or healthy controls (rₛ=−0.08416), remarkably, the ratio of these two related subsets were also highly different between the ME/CFS patients and the healthy controls (p<0.0001) (FIG. 8e)

### Machine Learning Analysis to Identify Predictive Immune Parameters for ME/CFS

Our immune profiling analysis identified many T cell subset parameters that were different in ME/CFS patients vs healthy controls. A total of 65 immune profile features were determined for the ME/CFS patients and healthy controls. These are tabulated in Table 2A below.

TABLE 2A
Immune profile features determined for the
ME/CFS patients and healthy controls

| No. | Feature |
|---|---|
| 1 | % CD3+ |
| 2 | % CD8+ |
| 3 | % CD4+ |
| 4 | CD4:CD8 |
| 5 | % CD4− CD8− |
| 6 | % CD4+ CD45RO+ CCR7+ |
| 7 | % CD4+ CD45RO− CCR7+ |
| 8 | % CD4+ CD45RO+ CCR7− |
| 9 | % CD4+ CD45RO− CCR7− |
| 10 | % CD8+ CD45RO+ CCR7+ |
| 11 | % CD8+ CD45RO− CCR7+ |
| 12 | % CD8+ CD45RO+ CCR7− |
| 13 | % CD8+ CD45RO− CCR7− |
| 14 | % CD45RO+ CD27+ (of DN) (d 0) |
| 15 | % CD45RO− CD27− (of DN) (d 0) |
| 16 | % CD45RO+ CD27− (of DN) (d 0) |
| 17 | % CD45RO+ CD27− (of CD 8+ MAIT) d 0 |
| 18 | % MAIT (of CD4+) (d 0) |
| 19 | % MAIT (of CD8+) (d 0) |
| 20 | % MAIT (of DN) (d 0) |
| 21 | % MAIT (of CD8+):% MAIT (of DN) (d 0) |
| 22 | CD4+ total memory % IL-17+ IFNγ+ (d 6) |
| 23 | CD4+ total memory % IL-17+ IFNγ− (d 6) |
| 24 | CD4+ total memory % IL-17+ (d 6) |
| 25 | CD4+ total memory % IFNγ+ (d 6) |
| 26 | CD4+ RO+ % IL-17+ IFNγ+ (of CCR6+) (d 6) |
| 27 | CD4+ RO+ % IL-17+ IFNγ− (of CCR6+) (d 6) |
| 28 | CD4+ RO+ % IL-17− IFNγ+ (of CCR6+) (d 6) |
| 29 | CD4+ RO+ % IL-17+ (of CCR6+) (d 6) |
| 30 | CD4+ RO+ % IFNγ+ (of CCR6+) (d 6) |
| 31 | % IFNγ+ (of memory CD4+) (d 6) |
| 32 | CD4+ CD45RO+ CCR6+ CD161+ % IL-17+ IFNγ+ (d 6) |
| 33 | CD4+ CD45RO+ CCR6+ CD161+ % IL-17+ IFNγ− (d 6) |
| 34 | CD4+ CD45RO+ CCR6+ CD161+ % IL-17− IFNγ+ (d 6) |
| 35 | CD4+ CD45RO+ CCR6+ CD161− % IL-17+ IFNγ+ (d 6) |
| 36 | CD4+ CD45RO+ CCR6+ CD161− % IL-17+ IFNγ− (d 6) |
| 37 | CD4+ CD45RO+ CCR6+ CD161− % IL-17− IFNγ+ (d 6) |
| 38 | % MAIT (of CD4+) (d 6) |
| 39 | % MAIT (of CD8+) (d 6) |
| 40 | % MAIT (of DN) (d 6) |
| 41 | % MAIT (of CD8+):% MAIT (of DN) (d 6) |
| 42 | % IL-17+ IFNγ+ (of CD8+ MAIT) (d 6) |
| 43 | % IFNγ+ (of CD8+ MAIT) (d 6) |
| 44 | % IL-17+ (of CD8+ MAIT) (d 6) |
| 45 | % TNFa (of CD8+ MAIT) (d 6) |
| 46 | % MAIT (of CD4+) (d 0:d 6) |
| 47 | % MAIT (of CD8+) (d 0:d 6) |
| 48 | % MAIT (of DN) (d 0:d 6) |
| 49 | % CCR6+ (of memory CD4+) (d 1) |
| 50 | CD4+ total memory % IL-17+ (d 1) |
| 51 | CD4+ RO+ % IL-17+ IFNγ+ (d 1) |
| 52 | CD4+ RO+ % IL-17+ IFNγ− (d 1) |
| 53 | CD4+ RO+ % IL-17+ (d 1) |
| 54 | CD4+ RO+ % IFNγ+ (d 1) |
| 55 | CD4+ RO+ % IL-17+ IFNγ+ (of CCR6+) (d 1) |
| 56 | CD4+ RO+ % IL-17+ IFNγ− (of CCR6+) (d 1) |
| 57 | CD4+ RO+ % IL-17+ (of CCR6+) (d 1) |
| 58 | CD4+ RO+ % IFNγ+ (of CCR6+) (d 1) |
| 59 | % IFNγ+ (of memory CD4+) (d 1) |
| 60 | % IFNγ+ (of CD8+ MAIT) (d 1) |
| 61 | % GranzymeA+ (of CD8+ MAIT) (d 1) |
| 62 | % Tregs (of naïve CD4+) (d 1) |
| 63 | % FOXP3+ (of naïve CD4+) (d 1) |
| 64 | % Tregs (of memory CD4+) (d 1) |
| 65 | % FOXP3+ (of memory CD4+) (d 1) |

To identify significant features, we performed a Student's t-test if the data in both groups was normally distributed; otherwise we performed the Mann-Whitney U test. From the total of 65 immune profile features, 40 features were identified as different after correction for a 5% false discovery rate, as shown in Table 3A below.

TABLE 3A
Immune profile features significantly different
between healthy controls and ME/CFS patients.

| No. | Feature | Raw p* | Adjusted p* |
|---|---|---|---|
| 1 | MAIT cells % of CD8+ to MAIT % of DN cells (d 6) | 1.48e−12 | 9.62e−11 |
| 2 | Granzyme A+ % of CD8+ MAIT cells (d 1) | 2.16e−09 | 7.04e−08 |
| 3 | IL-17+ % of CD4+CD45O+ memory (d 6) | 1.11e−07 | 2.40e−06 |
| 4 | IL-17+ IFNγ− of CD4+CD45RO+ memory (d 6) | 4.35e−07 | 7.07e−06 |
| 5 | IL-17+IFNγ+ % of CD4+CD45RO+ memory (d 6) | 8.70e−07 | 1.13e−05 |
| 6 | IL-17+ % of CD4+CD45RO+CCR6+ (d 6) | 1.31e−06 | 1.42e−05 |
| 7 | IL-17+IFNγ+ % of CD4+CD45RO+CCR6+ (d 6) | 1.70e−06 | 1.58e−05 |
| 8 | IFNγ+ % of CD4+CD45RO+ memory (d 6) | 7.53e−06 | 6.12e−05 |
| 9 | IFNγ+ % of CD8+ MAIT cells (d 1) | 1.01e−05 | 7.33e−05 |
| 10 | IL-17+IFNγ− % of CD4+CD45RO+CCR6+ (d 6) | 1.32e−05 | 7.36e−05 |
| 11 | MAIT cell ratio (d 0:d 6) % of CD8+ | 3.19e−05 | 0.00018 |
| 12 | IFNγ+ % of CD4+CD45RO+ memory (d 6) | 5.21e−05 | 0.00028 |
| 13 | IL-17+IFNγ+ % of CD4+CD45RO+CCR6+CD161− (d 6) | 9.69e−05 | 0.00048 |
| 14 | CD8+CD45RO+CCR7− % of CD8+ | 0.00011 | 0.00053 |
| 15 | Tregs % of naïve CD4+ (d 1) | 0.00019 | 0.00086 |
| 16 | CCR6+ % of memory CD4+ (d 1) | 0.00048 | 0.0019 |
| 17 | IFNγ+ % of CD4+CD45RO+CCR6+ (d 6) | 0.00078 | 0.0030 |
| 18 | IL-17+ % of CD4+CD45RO+CCR6+ (d 1) | 0.0019 | 0.0064 |
| 19 | IL-17+ % of CD4+CD45RO+ (d 1) | 0.0019 | 0.0064 |
| 20 | IFNγ+ % of memory CD4+ (d 1) | 0.0019 | 0.0064 |
| 21 | FOXP3+ % of memory CD4+ (d 1) | 0.0021 | 0.0066 |
| 22 | IL-17+IFNγ+ % of CD4+CD45RO+CCR6+ (d 1) | 0.0022 | 0.0067 |
| 23 | CD4+CD45RO+CCR6+CD161− % IL-17+IFNγ− (d 6) | 0.0027 | 0.0075 |
| 24 | CD45RO+CD27− % of CD8+ MAIT | 0.0028 | 0.0075 |
| 25 | CD8+ % of CD3+ | 0.0033 | 0.0086 |
| 26 | Tregs % of CD4+ memory (d 1) | 0.0043 | 0.011 |
| 27 | CD4+ to CD8+ T cell ratio | 0.0048 | 0.011 |
| 28 | IL-17+IFNγ− % of CD4+CD45RO+CCR6+ (d 1) | 0.005 | 0.011 |
| 29 | IL-17+IFNγ+ % of CD4+CD45RO+CCR6+CD161+ (d 6) | 0.0051 | 0.011 |
| 30 | CD4+ RO+ % IL-17− IFNγ+ (of CCR6+) (d 6) | 0.006 | 0.013 |
| 31 | MAIT ratio (d 0:d 6) % of CD4+ | 0.0092 | 0.019 |
| 32 | CD4+ % of CD3+ | 0.011 | 0.023 |
| 33 | IL-17+IFNγ+ % of CD8+ MAIT cells (d 6) | 0.012 | 0.023 |
| 34 | MAIT % of CD4+ (d 6) | 0.013 | 0.024 |
| 35 | MAIT % of CD8+ (d 6) | 0.013 | 0.024 |
| 36 | CD8+ MAIT ratio to DN MAIT cells (d 0) | 0.015 | 0.027 |
| 37 | IL-17IFNγ+ % of CD4+CD45RO+ (d 1) | 0.016 | 0.028 |
| 38 | IFNγ+ % of CD4+CD45RO+ (d 1) | 0.017 | 0.029 |
| 39 | CD45RO+CD27− % of DN T cells (d 0) | 0.018 | 0.030 |
| 40 | CD8+CD45RO−CCR7− % of CD8+ | 0.021 | 0.035 |

*Raw p-value: Student's t-test or Mann-Whitney U test. Adj. p-value: adjusted p-values after 5% false discovery rate correction.

While some of the individual features shown in Table 3 were highly significant, given the high variability and ranges in humans for immune parameters, on their own they would not have clinically relevant specificity and sensitivity to discriminate patients from healthy individuals. Therefore, we decided to use a classifier model using a machine learning algorithm called the random forest (RF) classifier (Wang, H., and Li, G. (2017). A Selective Review on Random Survival Forests for High Dimensional Data. Quant Biosci 36, 85-96. 10.22283/qbs.2017.36.2.85).

The RF classifier or algorithm is an ensemble method that depends on a large number of individual classification trees (Wang and Li, 2017; Huynh-Thu, V. A., and Geurts, P. (2019). Unsupervised Gene Network Inference with Decision Trees and Random Forests. Methods Mol Biol 1883, 195-215. 10.1007/978-1-4939-8882-2_8). Each classification tree emits a predicted class and the class with the most votes becomes the model prediction. The individual trees are designed using a randomly selected number of samples (sampling with replacement) and a randomly selected feature set to minimize correlation between trees. A large number of relatively uncorrelated classification trees (models) are combined to provide a robust classification of the individual sample (Aevermann, B. D., Novotny, M., Bakken, T., Miller, J. A., Diehl, A. D., Osumi-Sutherland, D., Lasken, R. S., Lein, E. S., and Scheuermann, R. H. (2018). Cell type discovery using single-cell transcriptomics: implications for ontological representation. Hum Mol Genet 27, R40-R47. 10.1093/hmg/ddy100).

As such, we implemented an RF model to classify ME/CFS patients and healthy controls using the immune profiling data. As discussed earlier in Materials and Methods, the RF classifier was trained using all 65 immune profile features, the 40 significantly different immune profile features, the top 10 significantly different immune profile features, and the top 10 immune profile features among the 40 significantly different features that received the highest importance score in the RF classifier model. Table 4A below presents the 10 immune profile features with the highest importance scores.

TABLE 4A
The 10 features with the highest importance score

| Immune Features | Importance score* | Adj. p value |
|---|---|---|
| MAIT % of CD8+ to MAIT % of DN ratio(d 6) | 0.210 | 9.62e−11 |
| GranzymeA+ % of CD8+ MAIT cells (d 1) | 0.126 | 7.04e−08 |
| MAIT % of CD8+ (d 0:d 6) | 0.106 | 0.00018 |
| IFNγ+ % of CD8+ MAIT cells (d 1) | 0.103 | 7.33e−05 |
| IL-17+IFNγ+ % of CD4+CD45RO+CCR6+CD161+ (d 6) | 0.086 | 0.011 |
| CD8+CD45RO−CCR7− % of CD8+ | 0.079 | 0.035 |
| IFNγ+ % of memory CD4+ (d 6) | 0.077 | 0.00028 |
| MAIT % of CD8+ to MAIT % of DN (d 0) | 0.072 | 0.027 |
| IL-17+IFNγ+ % of CD4+CD45RO+ CCR6+ (d 6) | 0.071 | 1.58e−05 |
| Tregs (Foxp3+Helios+) % of naïve CD4+ (d 1) | 0.069 | 0.00086 |

*Importance score determined in the RF model derived from the 40 significantly different features.

The performance of the RF was evaluated using a receiver operating characteristic (ROC) curve, which is created by plotting the true positive rate (TPR) against the false positive rate (FPR). The class prediction probability of a sample can be computed based on the proportion of votes obtained for that call. Given a threshold T for the probability, a sample is classified as an ME/CFS patient if the probability is higher than T and the ROC curve plots TPR against the FPR.

The area under the ROC curve which is denoted by AUC is equal to the probability that a randomly chosen positive instance will be ranked higher than a randomly chosen negative instance. A perfect classifier will have the maximal area under the curve of 1. The ROC curves of the RF classifier corresponding to 4 subsets of immune profile features are shown in FIG. 9. The AUC of the RF classifier using all 65 features is ˜0.93, meaning that there is a chance of 93% that the classifier will correctly distinguish between patients and healthy controls (FIG. 9 and Table 5). An RF classifier trained on the 40 significantly different features or on the top 10 features with the highest importance score among these 40 significantly different immune parameters had a slightly lower AUC scores (−0.92 and ˜0.88, respectively), whereas the RF classifier trained on the top 10 significantly different features had a lower AUC score (˜0.82) (FIG. 9 and Table 5).

Table 5 shows the sensitivity (recall), specificity, positive predictive value (precision), negative predictive value, accuracy, and F₁ score for the RF model using the various sets of features described above.

TABLE 5
Metrics of the RF classifier trained using different numbers of immune profile features.

| Number of | | | Positive | Negative | | | |
|---|---|---|---|---|---|---|---|
| features | Sensitivity | Specificity | pred. value | pred. value | Accuracy | F₁ score | AUC |
| 65 total set | 0.950 | 0.611 | 0.844 | 0.846 | 0.845 | 0.894 | 0.929 |
| 40 significant | 0.900 | 0.722 | 0.878 | 0.765 | 0.845 | 0.889 | 0.915 |
| 10 important | 0.925 | 0.556 | 0.822 | 0.769 | 0.810 | 0.871 | 0.879 |
| 10 significant | 0.825 | 0.722 | 0.868 | 0.650 | 0.793 | 0.846 | 0.815 |

Detailed explanation of the metrics presented in Table 5, and formulas to calculate them, are given in Materials and Methods. The rows present the metrics calculated for the RF classifier model obtained using: 1) all 65 immune profile features, 2) the 40 significantly different immune profile features, 3) the 10 immune profile features with the highest importance score among the 40 significantly different immune profile features, and 4) the top 10 significantly different immune profile features.

The machine learning classifier using immune parameters as features was able to identify the ME/CFS patients at a high sensitivity and accuracy when using all 65 features, all 40 significantly different features, and the 10 features among the 40 significantly different features that had the highest importance score. For all four classifier models, we observed a higher value of sensitivity than specificity, indicating that the proportion of patients correctly identified as ME/CFS patients is higher than healthy controls who are correctly identified as healthy. One reason for this could be related to parameters not included in training the RF classifier, such as age which causes the older individuals' immune profiles to become more similar to those of ME/CFS patients, and hence the RF classifier categorizes healthy controls as patients.

Currently, diagnosis of ME/CFS is based on clinical symptoms alone. The system and method disclosed herein permitting classification based on a patient's immune profile provides an additional tool that can aid better diagnosis of this clinical problem.

The disclosure herein include(s) at least the following aspects:

Aspect 1. A method for developing a predictive model for diagnosis of myalgic encephalomyelitis/chronic fatigue syndrome (ME/CFS) in a human comprising: receiving immune system data for each member of a population comprising healthy humans and humans with myalgic encephalomyelitis/chronic fatigue syndrome (ME/CFS); extracting a set of features from the immune system data; and training a machine learning algorithm using the set of features to classify a human as healthy or having ME/CFS to obtain a predictive model.

Aspect 2. The method of aspect 1, further comprising evaluating performance of the predictive model with a test set of immune system data for a population comprising healthy humans and humans with ME/CFS.

Aspect 3. The method of aspect 2, wherein performance is evaluated using sensitivity, specificity, accuracy, positive predictive value, negative predictive value, F₁ score, a receiver operating characteristic (ROC) curve, or a combination thereof.

Aspect 4. The method of any one of aspects 1 to 3, wherein the machine learning algorithm is a random forest classifier, a support vector machine, an artificial neural network, or a combination thereof.

Aspect 5. The method of any one of aspects 1 to 4, further comprising receiving other data for each human in the population; and wherein extracting a set of features from the immune system data comprises extracting a set of features from the immune system data and the other data, wherein the other data for each patient comprises clinical symptoms, demographic information, metabolic biomarkers, microbiome biomarkers, clinical history, genetics, or a combination thereof.

Aspect 6. The method of any one of aspects 1 to 5 wherein receiving immune system data comprises receiving data for at least one of the features listed in Table 2.

Aspect 7. The method of any one of aspects 1 to 6 wherein receiving immune system data comprises receiving data for at least the immune features in Table 4.

Aspect 8. The method of any one of aspects 1 to 6 wherein receiving immune system data comprises receiving data for at least immune features 1-10 in Table 3.

Aspect 9. The method of any one of aspects 1 to 6 wherein receiving immune system data comprises receiving data for at least the immune features in Table 3.

Aspect 10. The method of any one of aspects 1 to 9 wherein receiving immune system data comprises receiving data for all the immune profile features listed in the table of aspect 6

Aspect 11. A method for diagnosing myalgic encephalomyelitis/chronic fatigue syndrome (ME/CFS) in a subject, comprises: receiving immune system data of a subject; extracting a set of features from the immune system data; inputting the features to a machine-trained classifier, the machine trained classifier trained, at least in part, from training data comprising immune system data for a population comprising healthy humans and humans with myalgic encephalomyelitis/chronic fatigue syndrome (ME/CFS); classifying, by application of the machine-trained classifier to the features, the subject as being healthy or having ME/CFS; and outputting the classification.

Aspect 12. The method of aspect 11 wherein receiving immune system data comprises receiving data for at least one of the features listed in Table 2.

Aspect 13. The method of any one of aspects 11 to 12 wherein receiving immune system data comprises receiving data for at least the immune features in Table 4.

Aspect 14. The method of any one of aspects 11 to 12 wherein receiving immune system data comprises receiving data for at least immune features 1-10 in Table 3.

Aspect 15. The method of any one of aspects 11 to 12 wherein receiving immune system data comprises receiving data for at least the immune features in Table 3.

Aspect 16. The method of any one of aspects 11 to 15 wherein receiving immune system data comprises receiving data for all the immune features listed in the table of aspect 12.

Aspect 17. The method of any one of aspects 11 to 16 further comprising receiving other data for the subject, wherein the other data for the subject comprises clinical symptoms, demographic information, metabolic biomarkers, microbiome biomarkers, clinical history, genetics, or a combination thereof.

Aspect 18. The method of any one of aspects 11 to 17, wherein extracting a set of features from the immune system data comprises extracting a set of features from the immune system data and the other data.

Aspect 19. The method of any one of aspects 11 to 18, wherein the predictive model of the machine trained classifier has an AUC of at least 0.75.

Aspect 20. The method of any one of aspects 11 to 19 further comprising treating a subject classified as having ME/CFS with activity management, a prescription sleep medicine, a pain relieving drug, a pain management method, an antidepressant, an anti-anxiety drug, a stress management method, or a combination thereof.

Aspect 21. A system for diagnosing myalgic encephalomyelitis/chronic fatigue syndrome (ME/CFS) in a subject, comprising: a processor; and a memory storing computer executable instructions, which when executed by the processor cause the processor to perform operations comprising: receiving immune system data of a subject; extracting a set of features from the immune system data; inputting the features to a machine-trained classifier, the machine trained classifier trained, at least in part, from training data comprising immune system data for a population comprising healthy humans and humans with myalgic encephalomyelitis/chronic fatigue syndrome (ME/CFS); classifying, by application of the machine-trained classifier to the features, the subject as being healthy or having ME/CFS; and outputting the classification.

Aspect 22. A system for developing a predictive model for diagnosis of myalgic encephalomyelitis/chronic fatigue syndrome (ME/CFS) in a human comprising a processor; and a memory storing computer executable instructions, which when executed by the processor cause the processor to perform operations comprising: receiving immune system data for each member of a population comprising healthy humans and humans with ME/CFS; extracting a set of features from the immune system data; and training a machine learning algorithm using the set of features to classify a human as healthy or having ME/CFS to obtain a predictive model.

Aspect 23. The method or system of any one of the preceding claims wherein the immune system data received comprises measurements of immune system biomarkers in a blood sample from a member of the population.

Aspect 24. The method or system of any one of the preceding claims wherein the immune system biomarkers are determined by staining peripheral blood mononuclear cells (PBMCs) for intracellular proteins, cell surface proteins, or a combination thereof and detecting the stained PBMCs.

Aspect 25. The method or system of any one of the preceding claims wherein detecting the stained PBMCs is determined by flow cytometry.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “+10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. In a list of alternatively useable species, “a combination thereof” means that the combination can include a combination of at least one element of the list with one or more like elements not named.

Unless otherwise specified herein, any reference to standards, regulations, testing methods and the like, refer to the standard, regulation, guidance, or method that is in force at the time of filing of the present application.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method for developing a predictive model for diagnosis of myalgic encephalomyelitis/chronic fatigue syndrome (ME/CFS) in a human comprising:
receiving immune system data for each member of a population comprising healthy humans and humans with myalgic encephalomyelitis/chronic fatigue syndrome (ME/CFS);
extracting a set of features from the immune system data; and
training a machine learning algorithm using the set of features to classify a human as healthy or having ME/CFS to obtain a predictive model.

2. The method of claim 1, further comprising evaluating performance of the predictive model with a test set of immune system data for a population comprising healthy humans and humans with ME/CFS.

3. The method of claim 2, wherein performance is evaluated using sensitivity, specificity, accuracy, positive predictive value, negative predictive value, F₁ score, a receiver operating characteristic (ROC) curve, or a combination thereof.

4. The method of any one of claims 1 to 3, wherein the machine learning algorithm is a random forest classifier, a support vector machine, an artificial neural network, or a combination thereof.

5. The method of any one of claims 1 to 4, further comprising receiving other data for each human in the population; and
wherein extracting a set of features from the immune system data comprises extracting a set of features from the immune system data and the other data,
wherein the other data for each patient comprises clinical symptoms, demographic information, metabolic biomarkers, microbiome biomarkers, clinical history, genetics, or a combination thereof.

6. The method of any one of claims 1 to 5 wherein the extracted set of features comprises at least one of the features listed in the table below

| No. | Feature |
|---|---|
| 1 | % CD3+ |
| 2 | % CD8+ |
| 3 | % CD4+ |
| 4 | CD4:CD8 |
| 5 | % CD4− CD8− |
| 6 | % CD4+ CD45RO+ CCR7+ |
| 7 | % CD4+ CD45RO− CCR7+ |
| 8 | % CD4+ CD45RO+ CCR7− |
| 9 | % CD4+ CD45RO− CCR7− |
| 10 | % CD8+ CD45RO+ CCR7+ |
| 11 | % CD8+ CD45RO− CCR7+ |
| 12 | % CD8+ CD45RO+ CCR7− |
| 13 | % CD8+ CD45RO− CCR7− |
| 14 | % CD45RO+ CD27+ (of DN) (d 0) |
| 15 | % CD45RO− CD27− (of DN) (d 0) |
| 16 | % CD45RO+ CD27− (of DN) (d 0) |
| 17 | % CD45RO+ CD27− (of CD8+ MAIT) d 0 |
| 18 | % MAIT (of CD4+) (d 0) |
| 19 | % MAIT (of CD8+) (d 0) |
| 20 | % MAIT (of DN) (d 0) |
| 21 | % MAIT (of CD8+):% MAIT (of DN) (d 0) |
| 22 | CD4+ total memory % IL-17+ IFNγ+ (dy, where y = 3 to 14, preferably 3 to 10, more |
| | preferably 5-7, yet more preferably y = 6) |
| 23 | CD4+ total memory % IL-17+ IFNγ− (dy, where y = 3 to 14, preferably 3 to 10, more |
| | preferably 5-7, yet more preferably y = 6) |
| 24 | CD4+ total memory % IL-17+ (dy, where y = 3 to 14, preferably 3 to 10, more preferably |
| | 5-7, yet more preferably y = 6) |
| 25 | CD4+ total memory % IFNγ+ (dy, where y = 3 to 14, preferably 3 to 10, more preferably |
| | 5-7, yet more preferably y = 6) |
| 26 | CD4+ RO+ % IL-17+ IFNγ+ (of CCR6+) (dy, where y = 3 to 14, preferably 3 to 10, more |
| | preferably 5-7, yet more preferably y = 6) |
| 27 | CD4+ RO+ % IL-17+ IFNγ− (of CCR6+) (dy, where y = 3 to 14, preferably 3 to 10, more |
| | preferably 5-7, yet more preferably y = 6) |
| 28 | CD4+ RO+ % IL-17− IFNγ+ (of CCR6+) (dy, where y = 3 to 14, preferably 3 to 10, more |
| | preferably 5-7, yet more preferably y = 6) |
| 29 | CD4+ RO+ % IL-17+ (of CCR6+) (dy, where y = 3 to 14, preferably 3 to 10, more |
| | preferably 5-7, yet more preferably y = 6) |
| 30 | CD4+ RO+ % IFNγ+ (of CCR6+) (dy, where y = 3 to 14, preferably 3 to 10, more |
| | preferably 5-7, yet more preferably y = 6) |
| 31 | % IFNγ+ (of memory CD4+) (dy, where y = 3 to 14, preferably 3 to 10, more preferably |
| | 5-7, yet more preferably y = 6) |
| 32 | CD4+ CD45RO+ CCR6+ CD161+ % IL-17+ IFNγ+ (dy, where y = 3 to 14, preferably 3 |
| | to 10, more preferably 5-7, yet more preferably y = 6) |
| 33 | CD4+ CD45RO+ CCR6+ CD161+ % IL-17+ IFNγ− (dy, where y = 3 to 14, preferably 3 |
| | to 10, more preferably 5-7, yet more preferably y = 6) |
| 34 | CD4+ CD45RO+ CCR6+ CD161+ % IL-17− IFNγ+ (dy, where y = 3 to 14, preferably 3 |
| | to 10, more preferably 5-7, yet more preferably y = 6) |
| 35 | CD4+ CD45RO+ CCR6+ CD161− % IL-17+ IFNγ+ (dy, where y = 3 to 14, preferably 3 |
| | to 10, more preferably 5-7, yet more preferably y = 6) |
| 36 | CD4+ CD45RO+ CCR6+ CD161− % IL-17+ IFNγ− (dy, where y = 3 to 14, preferably 3 |
| | to 10, more preferably 5-7, yet more preferably y = 6) |
| 37 | CD4+ CD45RO+ CCR6+ CD161− % IL-17− IFNγ+ (dy, where y = 3 to 14, preferably 3 |
| | to 10, more preferably 5-7, yet more preferably y = 6) |
| 38 | % MAIT (of CD4+) (dy, where y = 3 to 14, preferably 3 to 10, more preferably 5-7, yet |
| | more preferably y = 6) |
| 39 | % MAIT (of CD8+) (dy, where y = 3 to 14, preferably 3 to 10, more preferably 5-7, yet |
| | more preferably y = 6) |
| 40 | % MAIT (of DN) (dy, where y = 3 to 14, preferably 3 to 10, more preferably 5-7, yet more |
| | preferably y = 6) |
| 41 | % MAIT (of CD8+):% MAIT (of DN) (dy, where y = 3 to 14, preferably 3 to 10, more |
| | preferably 5-7, yet more preferably y = 6) |
| 42 | % IL-17+ IFNγ+ (of CD8+ MAIT) (dy, where y = 3 to 14, preferably 3 to 10, more |
| | preferably 5-7, yet more preferably y = 6) |
| 43 | % IFNγ+ (of CD8+ MAIT) (dy, where y = 3 to 14, preferably 3 to 10, more preferably 5- |
| | 7, yet more preferably y = 6) |
| 44 | % IL-17+ (of CD8+ MAIT) (dy, where y = 3 to 14, preferably 3 to 10, more preferably 5- |
| | 7, yet more preferably y = 6) |
| 45 | % TNFa (of CD8+ MAIT) (dy, where y = 3 to 14, preferably 3 to 10, more preferably 5- |
| | 7, yet more preferably y = 6) |
| 46 | % MAIT (of CD4+) (d 0:dy, where y = 3 to 14, preferably 3 to 10, more preferably 5-7, |
| | yet more preferably y = 6) |
| 47 | % MAIT (of CD8+) (d 0:dy, where y = 3 to 14, preferably 3 to 10, more preferably 5-7, |
| | yet more preferably y = 6) |
| 48 | % MAIT (of DN) (d 0:dy, where y = 3 to 14, preferably 3 to 10, more preferably 5-7, yet |
| | more preferably y = 6) |
| 49 | % CCR6+ (of memory CD4+) (d 1) |
| 50 | CD4+ total memory % IL-17+ (d 1) |
| 51 | CD4+ RO+ % IL-17+ IFNγ+ (d 1) |
| 52 | CD4+ RO+ % IL-17+ IFNγ− (d 1) |
| 53 | CD4+ RO+ % IL-17+ (d 1) |
| 54 | CD4+ RO+ % IFNγ+ (d 1) |
| 55 | CD4+ RO+ % IL-17+ IFNγ+ (of CCR6+) (d 1) |
| 56 | CD4+ RO+ % IL-17+ IFNγ− (of CCR6+) (d 1) |
| 57 | CD4+ RO+ % IL-17+ (of CCR6+) (d 1) |
| 58 | CD4+ RO+ % IFNγ+ (of CCR6+) (d 1) |
| 59 | % IFNγ+ (of memory CD4+) (d 1) |
| 60 | % IFNγ+ (of CD8+ MAIT) (d 1) |
| 61 | % GranzymeA+ (of CD8+ MAIT) (d 1) |
| 62 | % Tregs (of naïve CD4+) (d 1) |
| 63 | % FOXP3+ (of naïve CD4+) (d 1) |
| 64 | % Tregs (of memory CD4+) (d 1) |
| 65 | % FOXP3+ (of memory CD4+) (d 1) |

7. The method of any one of claims 1 to 6 wherein the extracted set of features comprises at least the immune features in the table below.
MAIT % of CD8+ to MAIT % of DN ratio(dy, where y = 3
to 14, preferably 3 to 10, more preferably 5-7, yet more
preferably y = 6)
GranzymeA+ % of CD8+ MAIT (d 1)
MAIT % of CD8+ (d 0:dy, where y = 3 to 14, preferably 3
to 10, more preferably 5-7, yet more preferably y = 6)
ITNγ+ % of CD8+ MAIT (d 1)
IL-17+IFNγ+ % of CD4+CD45RO+CCR6+CD161+ (dy,
where y = 3 to 14, preferably 3 to 10, more preferably 5-7,
yet more preferably y = 6)
CD8+CD45RO−CCR7− % of CD8+
IFNγ+ % of memory CD4+ (dy, where y = 3 to 14, preferably
3 to 10, more preferably 5-7, yet more preferably y = 6)
MAIT % of CD8+ to MAIT % of DN (d 0)
IL-17+IFNγ+ % of CD4+CD45RO+ CCR6+ (dy, where y =
3 to 14, preferably 3 to 10, more preferably 5-7, yet more
preferably y = 6)
Tregs (Foxp3+Helios+) % of naïve CD4+ (d 1)

8. The method of any one of claims 1 to 6 wherein the extracted set of features comprises at least the immune features in the table below.
MAIT cells % of CD8+ to MAIT % of DN cells (dy, where y = 3 to 14,
preferably 3 to 10, more preferably 5-7, yet more preferably y = 6)
Granzyme A+ % of CD8+ MAIT cells (d 1)
IL-17+ % of CD4+CD45O+ memory (dy, where y = 3 to 14, preferably
3 to 10, more preferably 5-7, yet more preferably y = 6)
IL-17+ IFNγ− of CD4+CD45RO+ memory (dy, where y = 3 to 14,
preferably 3 to 10, more preferably 5-7, yet more preferably y = 6)
IL-17+IFNγ+ % of CD4+CD45RO+ memory (dy, where y = 3 to 14,
preferably 3 to 10, more preferably 5-7, yet more preferably y = 6)
IL-17+ % of CD4+CD45RO+CCR6+ (dy, where y = 3 to 14, preferably
3 to 10, more preferably 5-7, yet more preferably y = 6)
IL-17+IFNγ+ % of CD4+CD45RO+CCR6+ (dy, where y = 3 to 14,
preferably 3 to 10, more preferably 5-7, yet more preferably y = 6)
IFNγ+ % of CD4+CD45RO+ memory (dy, where y = 3 to 14, preferably
3 to 10, more preferably 5-7, yet more preferably y = 6)
IFNγ+ % of CD8+ MAIT cells (d 1)
IL-17+IFNγ− % of CD4+CD45RO+CCR6+ (dy, where y = 3 to 14,
preferably 3 to 10, more preferably 5-7, yet more preferably y = 6)

9. The method of any one of claims 1 to 6 wherein the extracted set of features comprises at least the immune features in the table below,
MAIT cells % of CD8+ to MAIT % of DN cells (dy, where y = 3 to 14,
preferably 3 to 10, more preferably 5-7, yet more preferably y = 6)
Granzyme A+ % of CD8+ MAIT cells (d 1)
IL-17+ % of CD4+CD45O+ memory (dy, where y = 3 to 14, preferably
3 to 10, more preferably 5-7, yet more preferably y = 6)
IL-17+ IFNγ− of CD4+CD45RO+ memory (dy, where y = 3 to 14,
preferably 3 to 10, more preferably 5-7, yet more preferably y = 6)
IL-17+IFNγ+ % of CD4+CD45RO+ memory (dy, where y = 3 to 14,
preferably 3 to 10, more preferably 5-7, yet more preferably y = 6)
IL-17+ % of CD4+CD45RO+CCR6+ (dy, where y = 3 to 14, preferably
3 to 10, more preferably 5-7, yet more preferably y = 6)
IL-17+IFNγ+ % of CD4+CD45RO+CCR6+ (dy, where y =
3 to 14, preferably 3 to 10, more preferably 5-7, yet more preferably
y = 6)
IFNγ+ % of CD4+CD45RO+ memory (dy, where y = 3 to 14,
preferably 3 to 10, more preferably 5-7, yet more preferably y = 6)
IPNγ+ % of CD8+ MAIT cells (d 1)
IL-17+IFNγ− % of CD4+CD45RO+CCR6+ (dy, where y =
3 to 14, preferably 3 to 10, more preferably 5-7, yet more preferably
y = 6)
MAIT cell ratio (d 0:dy, where y = 3 to 14, preferably 3 to 10, more
preferably 5-7, yet more preferably y = 6) % of CD8+
IFNγ+ % of CD4+CD45RO+ memory (dy, where y = 3 to 14,
preferably 3 to 10, more preferably 5-7, yet more preferably y = 6)
IL-17+IFNγ+ % of CD4+CD45RO+CCR6+CD161− (dy, where
y = 3 to 14, preferably 3 to 10, more preferably 5-7, yet more
preferably y = 6)
CD8+CD45RO+CCR7− % of CD8+
Tregs % of naïve CD4+ (d 1)
CCR6+ % of memory CD4+ (d 1)
IFNγ+ % of CD4+CD45RO+CCR6+ (dy, where y = 3 to 14,
preferably 3 to 10, more preferably 5-7, yet more preferably y = 6)
IL-17+ % of CD4+CD45RO+CCR6+ (d 1)
IL-17+ % of CD4+CD45RO+ (d 1)
IPNγ+ % of memory CD4+ (d 1)
FOXP3+ % of memory CD4+ (d 1)
IL-17+IFNγ+ % of CD4+CD45RO+CCR6+ (d 1)
CD4+CD45RO+CCR6+CD161− % IL-17+IPNγ− (dy, where
y = 3 to 14, preferably 3 to 10, more preferably 5-7, yet more
preferably y = 6)
CD45RO+CD27− % of CD8+ MAIT
CD8+ % of CD3+
Tregs % of CD4+ memory (d 1)
CD4+ to CD8+ T cell ratio
IL-17+IPNγ− % of CD4+CD45RO+CCR6+ (d 1)
IL-17+IFNγ7+ % of CD4+CD45RO+CCR6+CD161+ (dy, where
y = 3 to 14, preferably 3 to 10, more preferably 5-7, yet more
preferably y = 6)
CD4+ RO+ % IL-17− IFNγ+ (of CCR6+) (dy, where y =
3 to 14, preferably 3 to 10, more preferably 5-7, yet more preferably
y = 6)
MAIT ratio (d 0:dy, where y = 3 to 14, preferably 3 to 10, more
preferably 5-7, yet more preferably y = 6) % of CD4+
CD4+ % of CD3+
IL-17+IFNγ+ % of CD8+ MAIT cells (dy, where y = 3 to 14,
preferably 3 to 10, more preferably 5-7, yet more preferably y = 6)
MAIT % of CD4+ (dy, where y = 3 to 14, preferably 3 to 10, more
preferably 5-7, yet more preferably y = 6)
MAIT % of CD8+ (dy, where y = 3 to 14, preferably 3 to 10, more
preferably 5-7, yet more preferably y = 6)
CD8+ MAIT ratio to DN MAIT cells (d 0)
IL-17IFNγ+ % of CD4+CD45RO+ (d 1)
IFNγ+ % of CD4+CD45RO+ (d 1)
CD45RO+CD27− % of DN T cells (d 0)
CD8+CD45RO−CCR7− % of CD8+

10. The method of any one of claims 1 to 9 wherein the extracted set of features comprises all the immune profile features listed in the table of claim 6

11. A method for diagnosing myalgic encephalomyelitis/chronic fatigue syndrome (ME/CFS) in a subject, comprising:
receiving immune system data of a subject;
extracting a set of features from the immune system data;
inputting the features to classifier;
classifying, by application of the classifier to the features, the subject as being healthy or having ME/CFS; and
outputting the classification.

12. The method of claim 11 wherein the extracted set of features comprises at least one of the features listed in the table below.

| No. | Feature |
|---|---|
| 1 | % CD3+ |
| 2 | % CD8+ |
| 3 | % CD4+ |
| 4 | CD4:CD8 |
| 5 | % CD4− CD8− |
| 6 | % CD4+ CD45RO+ CCR7+ |
| 7 | % CD4+ CD45RO− CCR7+ |
| 8 | % CD4+ CD45RO+ CCR7− |
| 9 | % CD4+ CD45RO− CCR7− |
| 10 | % CD8+ CD45RO+ CCR7+ |
| 11 | % CD8+ CD45RO− CCR7+ |
| 12 | % CD8+ CD45RO+ CCR7− |
| 13 | % CD8+ CD45RO− CCR7− |
| 14 | % CD45RO+ CD27+ (of DN) (d 0) |
| 15 | % CD45RO− CD27− (of DN) (d 0) |
| 16 | % CD45RO+ CD27− (of DN) (d 0) |
| 17 | % CD45RO+ CD27− (of CD8+ MAIT) d 0 |
| 18 | % MAIT (of CD4+) (d 0) |
| 19 | % MAIT (of CD8+) (d 0) |
| 20 | % MAIT (of DN) (d 0) |
| 21 | % MAIT (of CD8+):% MAIT (of DN) (d 0) |
| 22 | CD4+ total memory % IL-17+ IFNγ+ (dy, where y = 3 to 14, preferably 3 to 10, more |
| | preferably 5-7, yet more preferably y = 6) |
| 23 | CD4+ total memory % IL-17+ IFNγ− (dy, where y = 3 to 14, preferably 3 to 10, more |
| | preferably 5-7, yet more preferably y = 6) |
| 24 | CD4+ total memory % IL-17+ (dy, where y = 3 to 14, preferably 3 to 10, more preferably |
| | 5-7, yet more preferably y = 6) |
| 25 | CD4+ total memory % IFNγ+ (dy, where y = 3 to 14, preferably 3 to 10, more preferably |
| | 5-7, yet more preferably y = 6) |
| 26 | CD4+ RO+ % IL-17+ IFNγ+ (of CCR6+) (dy, where y = 3 to 14, preferably 3 to 10, more |
| | preferably 5-7, yet more preferably y = 6) |
| 27 | CD4+ RO+ % IL-17+ IFNγ− (of CCR6+) (dy, where y = 3 to 14, preferably 3 to 10, more |
| | preferably 5-7, yet more preferably y = 6) |
| 28 | CD4+ RO+ % IL-17− IFNγ+ (of CCR6+) (dy, where y = 3 to 14, preferably 3 to 10, more |
| | preferably 5-7, yet more preferably y = 6) |
| 29 | CD4+ RO+ % IL-17+ (of CCR6+) (dy, where y = 3 to 14, preferably 3 to 10, more |
| | preferably 5-7, yet more preferably y = 6) |
| 30 | CD4+ RO+ % IFNγ+ (of CCR6+) (dy, where y = 3 to 14, preferably 3 to 10, more |
| | preferably 5-7, yet more preferably y = 6) |
| 31 | % IFNγ+ (of memory CD4+) (dy, where y = 3 to 14, preferably 3 to 10, more preferably |
| | 5-7, yet more preferably y = 6) |
| 32 | CD4+ CD45RO+ CCR6+ CD161+ % IL-17+ IFNγ+ (dy, where y = 3 to 14, preferably 3 |
| | to 10, more preferably 5-7, yet more preferably y = 6) |
| 33 | CD4+ CD45RO+ CCR6+ CD161+ % IL-17+ IFNγ− (dy, where y = 3 to 14, preferably 3 |
| | to 10, more preferably 5-7, yet more preferably y = 6) |
| 34 | CD4+ CD45RO+ CCR6+ CD161+ % IL-17− IFNγ+ (dy, where y = 3 to 14, preferably 3 |
| | to 10, more preferably 5-7, yet more preferably y = 6) |
| 35 | CD4+ CD45RO+ CCR6+ CD161− % IL-17+ IFNγ+ (dy, where y = 3 to 14, preferably 3 |
| | to 10, more preferably 5-7, yet more preferably y = 6) |
| 36 | CD4+ CD45RO+ CCR6+ CD161− % IL-17+ IFNγ− (dy, where y = 3 to 14, preferably 3 |
| | to 10, more preferably 5-7, yet more preferably y = 6) |
| 37 | CD4+ CD45RO+ CCR6+ CD161− % IL-17− IFNγ+ (dy, where y = 3 to 14, preferably 3 |
| | to 10, more preferably 5-7, yet more preferably y = 6) |
| 38 | % MAIT (of CD4+) (dy, where y = 3 to 14, preferably 3 to 10, more preferably 5-7, yet |
| | more preferably y = 6) |
| 39 | % MAIT (of CD8+) (dy, where y = 3 to 14, preferably 3 to 10, more preferably 5-7, yet |
| | more preferably y = 6) |
| 40 | % MAIT (of DN) (dy, where y = 3 to 14, preferably 3 to 10, more preferably 5-7, yet more |
| | preferably y = 6) |
| 41 | % MAIT (of CD8+):% MAIT (of DN) (dy, where y = 3 to 14, preferably 3 to 10, more |
| | preferably 5-7, yet more preferably y = 6) |
| 42 | % IL-17+ IFNγ+ (of CD8+ MAIT) (dy, where y = 3 to 14, preferably 3 to 10, more |
| | preferably 5-7, yet more preferably y = 6) |
| 43 | % IFNγ+ (of CD8+ MAIT) (dy, where y = 3 to 14, preferably 3 to 10, more preferably 5- |
| | 7, yet more preferably y = 6) |
| 44 | % IL-17+ (of CD8+ MAIT) (dy, where y = 3 to 14, preferably 3 to 10, more preferably 5- |
| | 7, yet more preferably y = 6) |
| 45 | % TNFa (of CD8+ MAIT) (dy, where y = 3 to 14, preferably 3 to 10, more preferably 5- |
| | 7, yet more preferably y = 6) |
| 46 | % MAIT (of CD4+) (d 0:dy, where y = 3 to 14, preferably 3 to 10, more preferably 5-7, |
| | yet more preferably y = 6) |
| 47 | % MAIT (of CD8+) (d0 :dy, where y = 3 to 14, preferably 3 to 10, more preferably 5-7, |
| | yet more preferably y = 6) |
| 48 | % MAIT (of DN) (d 0:dy, where y = 3 to 14, preferably 3 to 10, more preferably 5-7, yet |
| | more preferably y = 6) |
| 49 | % CCR6+ (of memory CD4+) (d 1) |
| 50 | CD4+ total memory % IL-17+ (d 1) |
| 51 | CD4+ RO+ % IL-17+ IFNγ+ (d 1) |
| 52 | CD4+ RO+ % IL-17+ IFNγ− (d 1) |
| 53 | CD4+ RO+ % IL-17+ (d 1) |
| 54 | CD4+ RO+ % IFNγ+ (d 1) |
| 55 | CD4+ RO+ % IL-17+ IFNγ+ (of CCR6+) (d 1) |
| 56 | CD4+ RO+ % IL-17+ IFNγ− (of CCR6+) (d 1) |
| 57 | CD4+ RO+ % IL-17+ (of CCR6+) (d 1) |
| 58 | CD4+ RO+ % IFNγ+ (of CCR6+) (d 1) |
| 59 | % IFNγ+ (of memory CD4+) (d 1) |
| 60 | % IFNγ+ (of CD8+ MAIT) (d 1) |
| 61 | % GranzymeA+ (of CD8+ MAIT) (d 1) |
| 62 | % Tregs (of naïve CD4+) (d 1) |
| 63 | % FOXP3+ (of naïve CD4+) (d 1) |
| 64 | % Tregs (of memory CD4+) (d 1) |
| 65 | % FOXP3+ (of memory CD4+) (d 1) |

13. The method of any one of claims 11 to 12 wherein the extracted set of features comprises at least the immune features in the table below.
MAIT % of CD8+ to MAIT % of DN ratio(dy, where y = 3 to 14,
preferably 3 to 10, more preferably 5-7, yet more preferably y = 6)
GranzymeA+ % of CD8+ MAIT (d 1)
MAIT % of CD8+ (d 0:dy, where y = 3 to 14, preferably 3 to 10,
more preferably 5-7, yet more preferably y = 6)
ITNγ+ % of CD8+ MAIT (d 1)
IL-17+IFNγ+ % of CD4+CD45RO+CCR6+CD161+ (dy,
where y = 3 to 14, preferably 3 to 10, more preferably 5-7,
yet more preferably y = 6)
CD8+CD45RO−CCR7− % of CD8+
IFNγ+ % of memory CD4+ (dy, where y = 3 to 14,
preferably 3 to 10, more preferably 5-7, yet more preferably y = 6)
MAIT % of CD8+ to MAIT % of DN (d 0)
IL-17+IFNγ+ % of CD4+CD45RO+ CCR6+ (dy, where y =
3 to 14, preferably 3 to 10, more preferably 5-7, yet more
preferably y = 6)
Tregs (Foxp3+Helios+) % of naïve CD4+ (d 1)

14. The method of any one of claims 11 to 12 wherein the extracted set of features comprises at least the immune features in the table below.
MAIT cells % of CD8+ to MAIT % of DN cells (dy, where y = 3 to 14,
preferably 3 to 10, more preferably 5-7, yet more preferably y = 6)
Granzyme A+ % of CD8+ MAIT cells (d 1)
IL-17+ % of CD4+CD45O+ memory (dy, where y = 3 to 14, preferably
3 to 10, more preferably 5-7, yet more preferably y = 6)
IL-17+ IFNγ− of CD4+CD45RO+ memory (dy, where y = 3 to 14,
preferably 3 to 10, more preferably 5-7, yet more preferably y = 6)
IL-17+IFNγ+ % of CD4+CD45RO+ memory (dy, where y = 3 to 14,
preferably 3 to 10, more preferably 5-7, yet more preferably y = 6)
IL-17+ % of CD4+CD45RO+CCR6+ (dy, where y = 3 to 14, preferably
3 to 10, more preferably 5-7, yet more preferably y = 6)
IL-17+IFNγ+ % of CD4+CD45RO+CCR6+ (dy, where y = 3 to 14,
preferably 3 to 10, more preferably 5-7, yet more preferably y = 6)
IFNγ+ % of CD4+CD45RO+ memory (dy, where y = 3 to 14, preferably
3 to 10, more preferably 5-7, yet more preferably y = 6)
IFNγ+ % of CD8+ MAIT cells (d 1)
IL-17+IFNγ− % of CD4+CD45RO+CCR6+ (dy, where y = 3 to 14,
preferably 3 to 10, more preferably 5-7, yet more preferably y = 6)

15. The method of any one of claims 11 to 12 wherein the extracted set of features comprises at least the immune features in the table below.
MAIT cells % of CD8+ to MAIT % of DN cells (dy, where y = 3 to 14,
preferably 3 to 10, more preferably 5-7, yet more preferably y = 6)
Granzyme A+ % of CD8+ MAIT cells (d 1)
IL-17+ % of CD4+CD45O+ memory (dy, where y = 3 to 14,
preferably 3 to 10, more preferably 5-7, yet more preferably y = 6)
IL-17+ IFNγ− of CD4+CD45RO+ memory (dy, where y =
3 to 14, preferably 3 to 10, more preferably 5-7, yet more preferably
y = 6)
IL-17+IFNγ+ % of CD4+CD45RO+ memory (dy, where y =
3 to 14, preferably 3 to 10, more preferably 5-7, yet more preferably
y = 6)
IL-17+ % of CD4+CD45RO+CCR6+ (dy, where y = 3 to 14,
preferably 3 to 10, more preferably 5-7, yet more preferably y = 6)
IL-17+IFNγ+ % of CD4+CD45RO+CCR6+ (dy, where y =
3 to 14, preferably 3 to 10, more preferably 5-7, yet more preferably
y = 6)
IFNγ+ % of CD4+CD45RO+ memory (dy, where y = 3 to 14,
preferably 3 to 10, more preferably 5-7, yet more preferably y = 6)
IFNγ+ % of CD8+ MAIT cells (d 1)
IL-17+IFNγ− % of CD4+CD45RO+CCR6+ (dy, where y =
3 to 14, preferably 3 to 10, more preferably 5-7, yet more preferably
y = 6)
MAIT cell ratio (d 0:dy, where y = 3 to 14, preferably 3 to 10,
more preferably 5-7, yet more preferably y = 6) % of CD8+
IFNγ+ % of CD4+CD45RO+ memory (dy, where y = 3 to 14,
preferably 3 to 10, more preferably 5-7, yet more preferably y = 6)
IL-17+IFNγ+ % of CD4+CD45RO+CCR6+CD161− (dy, where
y = 3 to 14, preferably 3 to 10, more preferably 5-7, yet more
preferably y = 6)
CD8+CD45RO+CCR7− % of CD8+
Tregs % of naïve CD4+ (d 1)
CCR6+ % of memory CD4+ (d 1)
IFNγ+ % of CD4+CD45RO+CCR6+ (dy, where y = 3 to 14,
preferably 3 to 10, more preferably 5-7, yet more preferably y = 6)
IL-17+ % of CD4+CD45RO+CCR6+ (d 1)
IL-17+ % of CD4+CD45RO+ (d 1)
IFNγ+ % of memory CD4+ (d 1)
FOXP3+ % of memory CD4+ (d 1)
IL-17+IFNγ+ % of CD4+CD45RO+CCR6+ (d 1)
CD4+CD45RO+CCR6+CD161− % IL−17+IPNγ− (dy,
where y = 3 to 14, preferably 3 to 10, more preferably 5-7, yet
more preferably y = 6)
CD45RO+CD27− % of CD8+ MAIT
CD8+ % of CD3+
Tregs % of CD4+ memory (d 1)
CD4+ to CD8+ T cell ratio
IL-17+IPNγ− % of CD4+CD45RO+CCR6+ (d 1)
IL-17+IFNΓ+ % of CD4+CD45RO+CCR6+CD161+ (dy, where
y = 3 to 14, preferably 3 to 10, more preferably 5-7, yet more
preferably y = 6)
CD4+ RO+ % IL-17− IFNγ+ (of CCR6+) (dy, where
y = 3 to 14, preferably 3 to 10, more preferably 5-7, yet more
preferably y = 6)
MAIT ratio (d 0:dy, where y = 3 to 14, preferably 3 to 10, more
preferably 5-7, yet more preferably y = 6) % of CD4+
CD4+ % of CD3+
IL-17+IFNγ+ % of CD8+ MAIT cells (dy, where y =
3 to 14, preferably 3 to 10, more preferably 5-7, yet more
preferably y = 6)
MAIT % of CD4+ (dy, where y = 3 to 14, preferably 3 to 10,
more preferably 5-7, yet more preferably y = 6)
MAIT % of CD8+ (dy, where y = 3 to 14, preferably 3 to 10,
more preferably 5-7, yet more preferably y = 6)
CD8+ MAIT ratio to DN MAIT cells (d 0)
IL-17IFNγ+ % of CD4+CD45RO+ (d 1)
IFNγ+ % of CD4+CD45RO+ (d 1)
CD45RO+CD27− % of DN T cells (d 0)
CD8+CD45RO−CCR7− % of CD8+

16. The method of any one of claims 11 to 15 wherein the extracted set of features comprises all the immune features listed in the table of claim 12.

17. The method of any one of claims 11 to 16 further comprising receiving other data for the subject, wherein the other data for the subject comprises clinical symptoms, demographic information, metabolic biomarkers, microbiome biomarkers, clinical history, genetics, or a combination thereof.

18. The method of any one of claims 11 to 17, wherein extracting a set of features from the immune system data comprises extracting a set of features from the immune system data and the other data.

19. The method of any one of claims 11 to 18, wherein the predictive model of the machine trained classifier has an AUC of at least 0.75.

20. The method of any one of claims 11 to 19 further comprising treating a subject classified as having ME/CFS with activity management, a prescription sleep medicine, a pain relieving drug, a pain management method, an antidepressant, an anti-anxiety drug, a stress management method, or a combination thereof.

21. A system for diagnosing myalgic encephalomyelitis/chronic fatigue syndrome (ME/CFS) in a subject, comprising:
a processor; and
a memory storing computer executable instructions, which when executed by the processor cause the processor to perform operations comprising:
receiving immune system data of a subject;
extracting a set of features from the immune system data;
inputting the features to a classifier);
classifying, by application of the classifier to the features, the subject as being healthy or having ME/CFS; and outputting the classification.

22. A system for developing a predictive model for diagnosis of myalgic encephalomyelitis/chronic fatigue syndrome (ME/CFS) in a human comprising:
a processor; and
a memory storing computer executable instructions, which when executed by the processor cause the processor to perform operations comprising:
receiving immune system data for each member of a population comprising healthy humans and humans with myalgic encephalomyelitis/chronic fatigue syndrome (ME/CFS);
extracting a set of features from the immune system data; and
training a machine learning algorithm using the set of features to classify a human as healthy or having ME/CFS to obtain a predictive model.

23. The method of claim 11 or the system of claim 21, wherein the classifier is a machine-trained classifier, the machine-trained classifier trained, at least in part, from training data comprising immune system data for a population comprising healthy humans and humans with myalgic encephalomyelitis/chronic fatigue syndrome (ME/CFS).

24. The method or system of any one of the preceding claims wherein the immune system data received comprises measurements of immune system biomarkers in a blood sample from a member of the population.

25. The method or system of any one of the preceding claims wherein the immune system biomarkers are determined by staining peripheral blood mononuclear cells (PBMCs) for intracellular proteins, cell surface proteins, or a combination thereof and detecting the stained PBMCs.

26. The method or system of any one of the preceding claims wherein detecting the stained PBMCs is determined by flow cytometry.
